SEQUENCE LISTING (4 sequences)

SEQ ID NO: 1 - DNA, 59 bp, synthetic construct
  misc_feature 1..59: note = Synthetic: Top strand
  modified_base 1: mod_base = OTHER; note = quencher Iowa Black
  modified_base 3: mod_base = OTHER; note = reporter fluorophore Fluorescein
  modified_base 16..19: mod_base = OTHER; note = protospacer adjacent motif (PAM) for Cas12a
  modified_base 20..39: mod_base = OTHER; note = spacer region hybridizing to a region of crRNA
  modified_base 56..59: mod_base = OTHER; note = Phosphorothioate linkage
  source 1..59: mol_type = other DNA
catggtgtcgacccatttcctggagccatctctctccttgctcgagacctctaagtatg

SEQ ID NO: 2 - DNA, 60 bp, synthetic construct
  misc_feature 1..60: note = Synthetic: Bottom strand
  modified_base 55..60: mod_base = OTHER; note = Phosphorothioate linkage
  source 1..60: mol_type = other DNA
gcatacttagaggtctcgagcaaggagagagatggctccaggaaatgggtcgacaccatg

SEQ ID NO: 3 - DNA, 55 bp, synthetic construct
  misc_feature 1..55: note = Synthetic: Top strand (no exonuclease protection)
  modified_base 1: mod_base = OTHER; note = quencher
  modified_base 3: mod_base = OTHER; note = fluorophore
  modified_base 16..19: mod_base = OTHER; note = protospacer adjacent motif (PAM) for Cas12a
  modified_base 20..39: mod_base = OTHER; note = protospacer adjacent motif (PAM) for Cas12a
  source 1..55: mol_type = other DNA
catggtgtcgacccatttcctggagccatctctctccttgctcgagacctctaag

SEQ ID NO: 4 - DNA, 60 bp, synthetic construct; identical to SEQ ID NO: 2
  misc_feature 1..60: note = Synthetic: Bottom strand (no exonuclease protection)
  source 1..60: mol_type = other DNA

(19) United States
(12) Patent Application Publication
(10) Pub. No.: US 2025/0019739 A1
(43) Pub. Date: Jan. 16, 2025

(54) EXONUCLEASE-COUPLED REAL-TIME ENDONUCLEASE ACTIVITY ASSAY

(71) Applicant: Caribou Biosciences, Inc., Berkeley, CA (US)

(72) Inventor: Stephen SMITH, Richmond, CA (US)

(73) Assignee: Caribou Biosciences, Inc., Berkeley, CA (US)

(21) Appl. No.: 18/704,687

(22) PCT Filed: Oct. 24, 2022
(86) PCT No.: PCT/US2022/078583

§ 371 Date: Apr. 25, 2024

Related U.S. Application Data
(60) Provisional application No. 63/272,091, filed on Oct. 26, 2021.

(51) Int. Cl. C12Q 1/34; C12Q 1/6818

(52) U.S. Cl. CPC C12Q 1/34; C12Q 1/6818

(57) ABSTRACT

The invention comprises a rapid in vitro method of assessing activity of an endonuclease, and a substrate therefor. Compositions, diagnostic methods, and kits are also disclosed.

### CROSS-REFERENCE TO RELATED APPLICATIONS

This application claims priority to the U.S. provisional application Ser. No. 63/272,091 filed on Oct. 26, 2021.

### FIELD OF THE INVENTION

The invention related to the field of nucleic acid-modifying enzymes and more specifically, to the field of developing and testing active endonucleases.

### STATEMENT REGARDING FEDERALLY SPONSORED RESEARCH

None.

### SEQUENCE LISTING

Placeholder.

### BACKGROUND OF THE INVENTION

Endonucleases are widely used enzymes in laboratory practice and molecular diagnostic industry. The process of designing and isolating new and improved endonucleases requires rapid and convenient methods of assessing endonuclease activity. The most widely used endonuclease assays are cumbersome and low throughput. Furthermore, the commonly used methods are not quantitative. There are no convention means to measure differences in activity between different sources, variants, or lots of endonucleases. Similarly, there are no easy or rapid means of assessing different targets so specificity of endonuclease could be accurately determined.

Many state-of-the-art endonuclease assays are unsuitable for use in crude cell lysates or partially purified protein preparations because host nucleases rapidly degrade the target DNA. There is an unmet need for a rapid endonuclease assay that would work in crude lysates and semi-crude elution fractions from early steps of a purification process. Such an assay would aid the efforts to improve enzyme purification and manufacturing and accelerate the overall enzyme engineering process.

### SUMMARY OF THE INVENTION

The instant invention provides methods, compositions, and kits for assessing endonuclease activity. The invention utilizes a double-stranded end-protected nucleic acid substrate labeled with a reporter fluorophore and a quencher fluorophore. An exonuclease hydrolyses the substrate and allows for fluorescence to occur only after the endonuclease cleaves the substrate.

In one embodiment, the invention is a nucleic acid substrate for detecting activity of an endonuclease comprising: a donor fluorophore and an acceptor fluorophore, said fluorophores forming a Fluorescence Resonance Energy Transfer (FRET) pair; at least one structure on at least one nucleic acid strand inhibiting cleavage of the substrate by an exonuclease; and a recognition sequence for an endonuclease. The structure inhibiting cleavage of the substrate by an exonuclease is selected from a structure at each 3′-end inhibiting cleavage by the 3′-5′exonuclease, a structure at each 5′-end inhibiting cleavage by the 5′-3′exonuclease, or both. The exonuclease is selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II. In some embodiments, the acceptor fluorophore is a quencher fluorophore. The donor fluorophore and the acceptor fluorophore may be placed between 1 and 12 nucleotides apart on the same strand of the substrate or on different strands of the substrate. In some embodiments, the substrate is formed by a single strand. In some embodiments, one of the donor fluorophore and the acceptor fluorophore is placed at or near a 5′-terminus of the substrate. In some embodiments, the donor fluorophore is selected from a group consisting of 5-carboxyfluorescein (5-FAM), 6-carboxyfluorescein (6-FAM), 2′,4′,1,4,-tetrachlorofluorescein (TET), 2′,4′,5′,7′, 1,4-hexachlorofluorescein (HEX), 2′,7′-dimethoxy-4′,5′-dichloro-6-carboxyfluorescein (JOE), coumarin dyes, Alexa Fluor dyes, IRDye 800CW, Cascade Blue, Pacific Blue, Pacific Orange, Texas Red, and BODIPY® dyes. In some embodiments, the acceptor fluorophore is selected from a group consisting of tetramethyl-6-carboxyrhodamine (TAMRA), tetrapropano-6-carboxyrhodamine (ROX), DABSYL, DABCYL (4-[[4-(dimethylamino)-phenyl]-azo]-benzoic acid), Cy5 and Cy5.5, anthraquinone dyes, nitrothiazole dyes, nitroimidazole dyes, LC-Red 610, LC-Red 640, LC-Red 705, JA286, DDQ-I, DDQ-II, QSY-7, QSY-21, IRDye QC1, Iowa Black FQ, Iowa Black RQ, HEX (hexachloro-fluorescein), TET (tetrachloro-fluorescein), JOE (5′-Dichloro-dimethoxy-fluorescein), BODIPY® dyes, Eclipse Quencher (4-[[2-chloro-4-nitro-phenyl]-azo]-aniline, BHQ-1 ([(4-(2-nitro-4-methyl-phenyl)-azo)-yl-((2-methoxy-5-methyl-phenyl)-azo)]-aniline), BHQ-2 ([(4-(1-nitro-phenyl)-azo)-yl-((2,5-dimethoxy-phenyl)-azo)]-aniline), and pyridinyl-isoquinoline-dione dyes. In some embodiments, the endonuclease is a nickase. In some embodiments, the exonuclease is selected from Exonuclease III, T5 Exonuclease, T7 Exonuclease, Lambda Exonuclease, and BAL31 Exonuclease. In some embodiments, the endonuclease is a nucleic acid-guided endonuclease, e.g., a CRISPR Class I (CASCADE) endonuclease. In some embodiments, the substrate comprises a protospacer adjacent motif (PAM). In some embodiments, the PAM consists of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In some embodiments, the nucleic acid-guided endonuclease is a CRISPR Class II endonuclease. In some embodiments, the endonuclease is a CRISPR Cas9 endonuclease. In some embodiments, the endonuclease is a CRISPR Cas12a endonuclease. In some embodiments, the PAM consists of a sequence selected from 5′-NGG-3′, 5′-NGGNG-3′, 5′-NNAAAAW-3′, 5′-NNNNGATT-3′, 5′-GNNNCNNA-3′, and 5′-NNNACA-3′. In some embodiments, the PAM consists of a sequence selected from 5′-TTN-3′, 5′-TTTN-3′ and 5′-TTTV-3′. In some embodiments the nucleic acid-guided endonuclease comprises an endonuclease and a nucleic acid targeting nucleic acid (NATNA), e.g., a CRISPR guide RNA selected from a single guide and a dual guide. In some embodiments, the NATNA comprises a crRNA and a tracrRNA. In some embodiments, the NATNA comprises a targeting region capable of hybridizing to a region in the substrate. In some embodiments, the NATNA is capable of interacting with the endonuclease. In some embodiments, the NATNA comprises DNA and RNA nucleotides. In some embodiments, the endonuclease is selected from a group consisting of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In some embodiments, the structure inhibiting cleavage of the substrate by an exonuclease is selected from a hairpin, a strand overhang, and a nucleic acid modification, e.g., one or more (5 or more) phosphorothioate linkages. In some embodiments, the endonuclease is a deoxyribonuclease, and the substrate contains DNA. In some embodiments, the endonuclease is a ribonuclease, and the substrate contains RNA. In some embodiments, the ribonuclease is selected from a ribozyme, a hammerhead ribozyme, a DNAzyme, a PNAzyme or an engineered endoribonuclease.

In one embodiment, the invention is a composition for detecting activity of an endonuclease comprising the nucleic acid substrate described above and the exonuclease, and optionally an endonuclease. In some embodiments, the endonuclease is a nickase. In some embodiments, the exonuclease is selected from Exonuclease III, T5 Exonuclease, T7 Exonuclease, Lambda Exonuclease, and BAL31 Exonuclease. In some embodiments, the exonuclease is capable of initiating hydrolysis from a nick. In some embodiments, the exonuclease is selected from T5 exonuclease, T7 exonuclease, Lambda exonuclease, Exonuclease III, and exonuclease Bal31. In some embodiments, the endonuclease is a nucleic acid-guided endonuclease. In some embodiments, the nucleic acid-guided endonuclease is a CRISPR Class I (CASCADE) endonuclease and the substrate comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′ 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In some embodiments, the nucleic acid-guided endonuclease is a CRISPR Cas9 endonuclease or a CRISPR Cas12a endonuclease and the substrate comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3′. In some embodiments, the nucleic acid-guided endonuclease comprises an endonuclease and a nucleic acid targeting nucleic acid (NATNA), e.g., a CRISPR guide RNA selected from a single guide and a dual guide. In some embodiments, the NATNA comprises a crRNA and a tracrRNA. In some embodiments, the NATNA comprises a targeting region capable of hybridizing to a region of the substrate. In some embodiments, the NATNA is capable of interacting with the endonuclease. In some embodiments, the NATNA comprises DNA and RNA nucleotides. In some embodiments, the endonuclease is selected from a group consisting of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In some embodiments, the structure inhibiting cleavage of the substrate by an exonuclease is selected from a hairpin, a strand overhang, and a nucleic acid modification, e.g., one or more (5 or more) phosphorothioate linkages.

In one embodiment, the invention is a method for detecting activity of an endonuclease comprising: contacting an endonuclease and an exonuclease with a reaction mixture comprising and a nucleic acid substrate comprising: a donor fluorophore and an acceptor fluorophore, said fluorophores forming a Fluorescence Resonance Energy Transfer (FRET) pair; at least one structure on at least one strand inhibiting cleavage of the substrate by the exonuclease; and a recognition sequence for the endonuclease and measuring fluorescence emitted by the reaction mixture, wherein a change in fluorescence indicates activity of the endonuclease. In some embodiments, the at least one structure inhibiting cleavage of the substrate by an exonuclease is selected from a structure at each 3′-end inhibiting cleavage by the 3′-5′exonuclease, a structure at each 5′-end inhibiting cleavage by the 5′-3′exonuclease, or both. In some embodiments, the exonuclease is selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II. In some embodiments, the reaction mixture is simultaneously contacted with the endonuclease and the exonuclease. In some embodiments, the reaction mixture is first contacted with the endonuclease, and subsequently is contacted with the exonuclease. In some embodiments, no purification steps are performed between contacting the reaction mixture with the endonuclease and contacting the reaction mixture with the exonuclease. In some embodiments, the endonuclease is a nucleic acid-guided endonuclease. In some embodiments, the nucleic acid-guided endonuclease is a CRISPR Class I (CASCADE) endonuclease and the substrate comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′ 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In some embodiments, the nucleic acid-guided endonuclease is a CRISPR Cas9 endonuclease or a CRISPR Cas12a endonuclease and the substrate comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3′. In some embodiments, the nucleic acid-guided endonuclease comprises an endonuclease and a nucleic acid targeting nucleic acid (NATNA), e.g., a CRISPR guide RNA selected from a single guide and a dual guide. In some embodiments, the NATNA comprises a crRNA and a tracrRNA. In some embodiments, the NATNA comprises a targeting region capable of hybridizing to a region of the substrate. In some embodiments, the NATNA is capable of interacting with the endonuclease. In some embodiments, the NATNA comprises DNA and RNA nucleotides. In some embodiments, the contacting comprises contacting a series of reaction mixtures comprising the same NATNA with a series of different endonucleases. In some embodiments, the contacting comprises contacting a series of reaction mixtures comprising the same endonuclease with a series of different NATNA. In some embodiments, the contacting comprises contacting a series of reaction mixtures comprising the same ingredients under different reaction conditions. In some embodiments, the contacting comprises contacting a series of reaction mixtures comprising the same ingredients with different isolates of the endonuclease. In some embodiments, the contacting comprises contacting a series of reaction mixtures comprising the same endonuclease with a series of different nucleic acid substrates comprising different sequences. In some embodiments, the endonuclease is selected from a group consisting of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In some embodiments, the structure inhibiting cleavage of the substrate by an exonuclease is selected from a hairpin, a strand overhang, and a nucleic acid modification, e.g., one or more (5 or more) phosphorothioate linkages. In some embodiments, the nucleic acid substrate or the endonuclease are in an unpurified form.

In one embodiment, the invention is a kit for detecting activity of an endonuclease comprising: a nucleic acid substrate comprising: a donor fluorophore and an acceptor fluorophore, said fluorophores forming a Fluorescence Resonance Energy Transfer (FRET) pair; at least one structure on at least one strand inhibiting cleavage of the substrate by an exonuclease; and a recognition sequence for an endonuclease; and the exonuclease. In some embodiments, the at least one structure inhibiting cleavage of the substrate by an exonuclease is selected from a structure at each 3′-end inhibiting cleavage by the 3′-5′exonuclease, a structure at each 5′-end inhibiting cleavage by the 5′-3′exonuclease, or both. In some embodiments, the exonuclease is selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II. In some embodiments, the endonuclease to be tested is a nucleic acid-guided endonuclease. In some embodiments, the nucleic acid-guided endonuclease is a CRISPR Class I (CASCADE) endonuclease and the substrate comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In some embodiments, the nucleic acid-guided endonuclease is a CRISPR Cas9 endonuclease or a CRISPR Cas12a endonuclease and the substrate comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3′. In some embodiments, the kit further comprises a nucleic acid targeting nucleic acid (NATNA) capable of forming a complex with the endonuclease to be tested. In some embodiments, the NATNA is a CRISPR guide RNA selected from a single guide and a dual guide. In some embodiments, the NATNA comprises a crRNA and a tracrRNA. In some embodiments, the NATNA comprises a targeting region capable of hybridizing to a region of the substrate. In some embodiments, the NATNA is capable of interacting with the endonuclease. In some embodiments, the NATNA comprises DNA and RNA nucleotides. In some embodiments, the endonuclease is selected from a group consisting of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In some embodiments, the structure inhibiting cleavage of the substrate by an exonuclease is selected from a hairpin, a strand overhang, and a nucleic acid modification, e.g., one or more (5 or more) phosphorothioate linkages.

In one embodiment, the invention is an apparatus for detecting activity of an endonuclease with the substrate of claim 1 comprising: a reaction chamber for performing enzymatic reactions and a fluorescence detector.

In one embodiment, the invention is a method for detecting the presence of a target nucleic acid in a sample, the method comprising: contacting a sample with a reaction mixture comprising an endonuclease, an exonuclease and a nucleic acid probe capable of hybridizing to a target nucleic acid, the probe comprising: a donor fluorophore and an acceptor fluorophore, said fluorophores forming a Fluorescence Resonance Energy Transfer (FRET) pair; at least one structure inhibiting cleavage of the probe by the exonuclease; and a recognition sequence for the endonuclease and measuring fluorescence emitted by the reaction mixture, wherein a change in fluorescence indicates the presence of the target nucleic acid in the sample. In some embodiments, the at least one structure inhibiting cleavage of the probe by an exonuclease is selected from a structure at each 3′-end inhibiting cleavage by the 3′-5′exonuclease, a structure at each 5′-end inhibiting cleavage by the 5′-3′exonuclease, or both. In some embodiments, the exonuclease is selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II. In some embodiments, the target nucleic acid is selected from a sequence characteristic of a bacterium, a sequence characteristic of a virus, a sequence characteristic of a parasite, and a patient's sequence characteristic of a patient's disease or condition. In some embodiments, the endonuclease is a nucleic acid-guided endonuclease. In some embodiments, the endonuclease is a CRISPR Class I (CASCADE) endonuclease and the probe comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In some embodiments, the endonuclease is a CRISPR Cas9 endonuclease or a CRISPR Cas12a endonuclease and the probe comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3′. In some embodiments, the nucleic acid-guided endonuclease comprises an endonuclease and a nucleic acid targeting nucleic acid (NATNA), e.g., a CRISPR guide RNA selected from a single guide and a dual guide. In some embodiments, the NATNA comprises a crRNA and a tracrRNA. In some embodiments, the NATNA comprises a targeting region capable of hybridizing to a region of the probe. In some embodiments, the NATNA is capable of interacting with the endonuclease. In some embodiments, the NATNA comprises DNA and RNA nucleotides. In some embodiments, the endonuclease is selected from a group consisting of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In some embodiments, the structure inhibiting cleavage of the probe by an exonuclease is selected from a hairpin, a strand overhang, and a nucleic acid modification, e.g., one or more (5 or more) phosphorothioate linkages. In some embodiments, the sample comprises a crude preparation of nucleic acids.

In one embodiment, the invention is a method for detecting the presence of two or more target nucleic acids in a sample, the method comprising: contacting a sample with a reaction mixture comprising an endonuclease, an exonuclease and two or more nucleic acid probes, each capable of hybridizing to two or more target nucleic acids, each probe comprising: a donor fluorophore and an acceptor fluorophore, said fluorophores forming a Fluorescence Resonance Energy Transfer (FRET) pair; at least one structure inhibiting cleavage of the probes by the exonuclease; and a recognition sequence for the endonuclease and measuring fluorescence emitted by the reaction mixture, wherein a change in fluorescence indicates the presence of the target nucleic acid in the sample. In some embodiments, the two or more nucleic acid probes comprises at least one different fluorophore. In some embodiments, all of the two or more nucleic acid probes comprise the same fluorophore. In some embodiments, the at least one structure inhibiting cleavage of the probes by an exonuclease is selected from a structure at each 3′-end inhibiting cleavage by the 3′-5′exonuclease, a structure at each 5′-end inhibiting cleavage by the 5′-3′exonuclease, or both. In some embodiments, the exonuclease is selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II. In some embodiments, the two or more target nucleic acids are selected from a sequence characteristic of a bacterium, a sequence characteristic of a virus, a sequence characteristic of a parasite, and a patient's sequence characteristic of a patient's disease or condition. In some embodiments, the endonuclease is a nucleic acid-guided endonuclease. In some embodiments, the endonuclease is a CRISPR Class I (CASCADE) endonuclease and the substrate comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In some embodiments, the nucleic acid-guided endonuclease is a CRISPR Cas9 endonuclease or a CRISPR Cas12a endonuclease and the substrate comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3′. In some embodiments, the nucleic acid-guided endonuclease comprises an endonuclease and a nucleic acid targeting nucleic acid (NATNA), e.g., a CRISPR guide RNA selected from a single guide and a dual guide. In some embodiments, the NATNA comprises a crRNA and a tracrRNA. In some embodiments, at least one NATNA is used for each of the two or more probes and each NATNA comprises a targeting region capable of hybridizing to a region of at least one probe. In some embodiments, the NATNA is capable of interacting with the endonuclease. In some embodiments, the NATNA comprises DNA and RNA nucleotides. In some embodiments, the endonuclease is selected from a group consisting of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In some embodiments, the structure inhibiting cleavage of the probe by an exonuclease is selected from a hairpin, a strand overhang, and a nucleic acid modification, e.g., one or more (5 or more) phosphorothioate linkages. In some embodiments, the sample comprises a crude preparation of nucleic acids.

In one embodiment, the invention is a method for detecting the presence of a target nucleic acid in a sample, the method comprising: attaching to a target nucleic acid in a sample: a donor fluorophore and an acceptor fluorophore, said fluorophores forming a Fluorescence Resonance Energy Transfer (FRET) pair; at least one structure inhibiting cleavage by an exonuclease; and a recognition sequence for the endonuclease, thereby forming a modified nucleic acid, and contacting the sample with the endonuclease and the exonuclease; measuring fluorescence emitted by the reaction mixture, wherein a change in fluorescence indicates the presence of the target nucleic acid in the sample. In some embodiments, the at least one structure inhibiting cleavage by an exonuclease is selected from a structure at each 3′-end inhibiting cleavage by the 3′-5′exonuclease, a structure at each 5′-end inhibiting cleavage by the 5′-3′exonuclease, or both. In some embodiments, the exonuclease is selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II. In some embodiments, the attaching is via ligation of adaptors including the donor fluorophore, the acceptor fluorophore and the structure inhibiting cleavage by an exonuclease. In some embodiments, prior to attaching, the target nucleic acid is amplified by PCR. In some embodiments, attaching is via one or more rounds of extension with amplification primers comprising a donor fluorophore and an acceptor fluorophore, said fluorophores forming a Fluorescence Resonance Energy Transfer (FRET) pair; a structure inhibiting cleavage by an exonuclease. In some embodiments, the target nucleic acid is selected from a sequence characteristic of a bacterium, a sequence characteristic of a virus, a sequence characteristic of a parasite, and a patient's sequence characteristic of a patient's disease or condition. In some embodiments, the endonuclease is a nucleic acid-guided endonuclease. In some embodiments, the endonuclease is a CRISPR Class I (CASCADE) endonuclease and the target nucleic acid or the modified target nucleic acid comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In some embodiments, the endonuclease is a CRISPR Cas9 endonuclease or a CRISPR Cas12a endonuclease and the target nucleic acid or the modified target nucleic acid comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3′. In some embodiments, the nucleic acid-guided endonuclease comprises an endonuclease and a nucleic acid targeting nucleic acid (NATNA), e.g., a CRISPR guide RNA selected from a single guide and a dual guide. In some embodiments, the NATNA comprises a crRNA and a tracrRNA. In some embodiments, the NATNA comprises a targeting region capable of hybridizing to a target nucleic acid or a modified target nucleic acid. In some embodiments, the NATNA is capable of interacting with the endonuclease. In some embodiments, the NATNA comprises DNA and RNA nucleotides. In some embodiments, the endonuclease is selected from a group consisting of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In some embodiments, the structure inhibiting cleavage by an exonuclease is selected from a hairpin, a strand overhang, and a nucleic acid modification, e.g., one or more (5 or more) phosphorothioate linkages. In some embodiments, the sample comprises a crude preparation of nucleic acids.

In one embodiment, the invention is a kit for performing a diagnostic procedure according to the method of claim 116 comprising: an endonuclease and a nucleic acid probe capable of hybridizing to a target nucleic acid, the probe comprising: a donor fluorophore and an acceptor fluorophore, said fluorophores forming a Fluorescence Resonance Energy Transfer (FRET) pair, at least one structure inhibiting cleavage of the probe by the exonuclease; and a recognition sequence for the endonuclease and measuring fluorescence emitted by the reaction mixture, wherein a change in fluorescence indicates the presence of the target nucleic acid in the sample, and wherein the target nucleic acid is selected from a sequence characteristic of a bacterium, a sequence characteristic of a virus, a sequence characteristic of a parasite, and a patient's sequence characteristic of a patient's disease or condition. In some embodiments, the at least one structure inhibiting cleavage of the probe by an exonuclease is selected from a structure at each 3′-end inhibiting cleavage by the 3′-5′exonuclease, a structure at each 5′-end inhibiting cleavage by the 5′-3′exonuclease, or both. In some embodiments, the kit further comprises an exonuclease selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II. In some embodiments, the endonuclease is a nucleic acid-guided endonuclease. In some embodiments, the nucleic acid-guided endonuclease is a CRISPR Class I (CASCADE) endonuclease and the probe comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In some embodiments, the nucleic acid-guided endonuclease is a CRISPR Cas9 endonuclease or a CRISPR Cas12a endonuclease and the probe comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3′. In some embodiments, the nucleic acid-guided endonuclease is a CRISPR endonuclease in complex with a nucleic acid targeting nucleic acid (NATNA), and the NATNA is a CRISPR guide RNA selected from a single guide and a dual guide. In some embodiments, the NATNA comprises a crRNA and a tracrRNA. In some embodiments, the NATNA is capable of interacting with the endonuclease. In some embodiments, the NATNA comprises DNA and RNA nucleotides. In some embodiments, the endonuclease is selected from a group consisting of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In some embodiments, the structure inhibiting cleavage by an exonuclease is selected from a hairpin, a strand overhang, and a nucleic acid modification, e.g., one or more (5 or more) phosphorothioate linkages.

In one embodiment, the invention is a kit for detecting the presence of a target nucleic acid in a sample, the method comprising one or more oligonucleotides capable of being attached to a target nucleic acid to form a modified nucleic acid, the oligonucleotides comprising: a donor fluorophore and an acceptor fluorophore, said fluorophores forming a Fluorescence Resonance Energy Transfer (FRET) pair; at least one structure inhibiting cleavage by an exonuclease; wherein the modified nucleic acid comprises a recognition sequence for an endonuclease, and the endonuclease, and wherein the target nucleic acid is selected from a sequence characteristic of a bacterium, a sequence characteristic of a virus, a sequence characteristic of a parasite, and a patient's sequence characteristic of a patient's disease or condition. In some embodiments, the at least one structure inhibiting cleavage by an exonuclease is selected from a structure at each 3′-end inhibiting cleavage by the 3′-5′exonuclease, a structure at each 5′-end inhibiting cleavage by the 5′-3′exonuclease, or both. In some embodiments, the exonuclease is selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II. In some embodiments, the attaching is via ligation of the oligonucleotides to the target nucleic acid and the kit optionally includes a ligase. In some embodiments, the attaching is via one or more rounds of extension with the oligonucleotides acting as amplification primers and the kit optionally includes reagents for performing the amplification. In some embodiments, the endonuclease is a nucleic acid-guided endonuclease. In some embodiments, the endonuclease is a CRISPR Class I (CASCADE) endonuclease and the target nucleic acid or the modified target nucleic acid comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In some embodiments, the endonuclease is a CRISPR Cas9 endonuclease or a CRISPR Cas12a endonuclease and the target nucleic acid or the modified target nucleic acid comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3′. In some embodiments, the nucleic acid-guided endonuclease comprises an endonuclease and a nucleic acid targeting nucleic acid (NATNA), e.g., a CRISPR guide RNA selected from a single guide and a dual guide. In some embodiments, the NATNA comprises a crRNA and a tracrRNA. In some embodiments, the NATNA comprises a targeting region capable of hybridizing to a target nucleic acid or a modified target nucleic acid. In some embodiments, the NATNA is capable of interacting with the endonuclease. In some embodiments, the NATNA comprises DNA and RNA nucleotides. In some embodiments, the endonuclease is selected from a group consisting of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In some embodiments, the structure inhibiting cleavage by an exonuclease is selected from a hairpin, a strand overhang, and a nucleic acid modification, e.g., one or more (5 or more) phosphorothioate linkages.

In one embodiment, the invention is a method for optimizing endonuclease digestion reactions, the method comprising: preparing a series of reaction mixture with an exonuclease and a nucleic acid substrate comprising: a donor fluorophore and an acceptor fluorophore, said fluorophores forming a Fluorescence Resonance Energy Transfer (FRET) pair; at least one structure on at least one strand inhibiting cleavage of the substrate by the exonuclease; and a recognition sequence for an endonuclease; contacting each of the series of reaction mixtures with different amounts of the endonuclease; measuring fluorescence emitted by the reaction mixture, wherein a change in fluorescence indicates activity of the endonuclease; selecting the amount of endonuclease yielding the highest fluorescence of the reaction mixture, or the highest rate of increase of fluorescence of the reaction mixture as the optimal endonuclease concentration. In some embodiments, the structure inhibiting cleavage of the substrate by an exonuclease is selected from a structure at each 3′-end inhibiting cleavage by the 3′-5′exonuclease, a structure at each 5′-end inhibiting cleavage by the 5′-3′exonuclease, or both. In some embodiments, the exonuclease is selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II. In some embodiments, the reaction mixture is simultaneously contacted with the endonuclease and the exonuclease. In some embodiments, the reaction mixture is first contacted with the endonuclease, and subsequently is contacted with the exonuclease. In some embodiments, no purification steps are performed between contacting the reaction mixture with the endonuclease and contacting the reaction mixture with the exonuclease.

In some embodiments, the endonuclease is a nucleic acid-guided endonuclease such as a CRISPR Class I (CASCADE) endonuclease and the substrate comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′, or a CRISPR Cas9 endonuclease or a CRISPR Cas12a endonuclease and the substrate comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3′.

In some embodiments, the nucleic acid-guided endonuclease comprises an endonuclease and a nucleic acid targeting nucleic acid (NATNA) e.g., a CRISPR guide RNA selected from a single guide and a dual guide. In some embodiments, the NATNA comprises a crRNA and a tracrRNA. In some embodiments, the NATNA comprises a targeting region capable of hybridizing to a region of the substrate. In some embodiments, the NATNA is capable of interacting with the endonuclease. In some embodiments, the NATNA comprises DNA and RNA nucleotides.

In some embodiments, the endonuclease is selected from a group consisting of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In some embodiments, the structure inhibiting cleavage of the substrate by an exonuclease is selected from a hairpin, a strand overhang, and a nucleic acid modification. In some embodiments, the nucleic acid modification comprises one or more phosphorothioate linkages. In some embodiments, the nucleic acid modification comprises 5 or more phosphorothioate linkages.

In one embodiment, the invention is a method for optimizing CRISPR endonuclease digestion reactions, the method comprising: preparing a series of reaction mixture with a CRISPR endonuclease, an exonuclease and a nucleic acid substrate comprising: a donor fluorophore and an acceptor fluorophore, said fluorophores forming a Fluorescence Resonance Energy Transfer (FRET) pair; at least one structure on at least one strand inhibiting cleavage of the substrate by the exonuclease; and a recognition sequence for an endonuclease; contacting each of the series of nucleic acid targeting nucleic acids (NATNAs); measuring fluorescence emitted by the reaction mixture, wherein a change in fluorescence indicates activity of the endonuclease; and selecting the NATNA yielding the highest fluorescence of the reaction mixture, or the highest rate of increase of fluorescence of the reaction mixture as the optimal NATNA. In some embodiments, the at least one structure inhibiting cleavage of the substrate by an exonuclease is selected from a structure at each 3′-end inhibiting cleavage by the 3′-5′exonuclease, a structure at each 5′-end inhibiting cleavage by the 5′-3′exonuclease, or both. In some embodiments, the exonuclease is selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II. In some embodiments, the reaction mixture is simultaneously contacted with the endonuclease and the exonuclease. In some embodiments, the reaction mixture is first contacted with the endonuclease, and subsequently is contacted with the exonuclease. In some embodiments,

no purification steps are performed between contacting the reaction mixture with the endonuclease and contacting the reaction mixture with the exonuclease.

In some embodiments, the CRISPR endonuclease is a Class I (CASCADE) endonuclease and the substrate comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In some embodiments, the CRISPR endonuclease is a Cas9 endonuclease or a Cas12a endonuclease and the substrate comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3′. In some embodiments, the NATNA is a CRISPR guide RNA selected from a single guide and a dual guide. In some embodiments, the NATNA comprises a crRNA and a tracrRNA. In some embodiments, the NATNA comprises a targeting region capable of hybridizing to a region of the substrate. In some embodiments, the NATNA comprises DNA and RNA nucleotides.

In some embodiments, the structure inhibiting cleavage of the substrate by an exonuclease is selected from a hairpin, a strand overhang, and a nucleic acid modification. In some embodiments, the nucleic acid modification comprises one or more phosphorothioate linkages. In some embodiments, the nucleic acid modification comprises 5 or more phosphorothioate linkages.

### BRIEF DESCRIPTION OF THE DRAWINGS

FIG. 1 is a diagram of the substrate and the method according to the invention.

FIG. 2 illustrates experimental validation of the utility of an exonuclease coupled fluorescent detection (Example 2).

FIG. 3 illustrates cleavage of the substrate (FIG. 1) by the Cas12a endonuclease (Example 3).

FIG. 4 illustrates experimental determination of the linear range of the assay with respect to Cas12a RNP concentration (Example 4).

FIG. 5 illustrates experimental confirmation that the exonuclease, not the Cas12a hydrolyzes the DNA (Example 5).

FIG. 6 illustrates experimental determination of the linear range of the assay with respect to DNA substrate concentration (Example 6).

FIG. 7 is a detailed diagram of the FAM-labeled substrate for CRISPR endonucleases.

FIG. 8 illustrates application of the exonuclease assay to cleavage of the FAM-labeled substrate (FIG. 7) by the Cas12a endonuclease (Example 9).

FIG. 9 illustrates application of the exonuclease assay to cleavage of the TAMRA-labeled substrate by the Cas12a endonuclease (Example 10).

FIG. 10 is a diagram of FAM-labeled substrates with different placements of the FAM fluorophore.

FIG. 11 illustrates application of the exonuclease assay to cleavage of the various FAM-labeled substrates (FIG. 10) by the Cas12a endonuclease (Example 11).

FIG. 12 is the summary of the data shown in FIG. 11.

FIG. 13 shown the rate of change in fluorescence of the reaction mixtures (Example 11).

FIG. 14 illustrates titration of the exonuclease in the assay applied to cleavage of the non-target FAM substrate by the Cas12a endonuclease (Example 12).

FIG. 15 illustrates application of the exonuclease assay to cleavage of the FAM-labeled substrate by the Cas9 endonuclease (Example 13).

FIG. 16 illustrates application of the exonuclease assay to cleavage of the FAM-labeled substrate by restriction endonucleases (Example 14).

### DETAILED DESCRIPTION OF THE INVENTION

### Definitions

The following definitions are provided to aid in understanding of the disclosure.

The term “endonuclease” refers to an enzyme catalyzing the hydrolysis of a phosphodiester bond between two nucleoside residues within a polynucleotide (DNA or RNA) wherein neither nucleoside residue is a terminal one.

The term “exonuclease” refers to an enzyme catalyzing the hydrolysis of a phosphodiester bond between a terminal nucleoside residue and a penultimate nucleoside residue within a polynucleotide (DNA or RNA). Exonucleases can be processive or capable of step-wise removal of multiple nucleoside residues from an end of a nucleic acid strand.

The term “CRISPR repeat” or “CRISPR repeat sequence” refers to a minimum CRISPR repeat sequence.

The term “endoribonuclease” refers to an enzyme catalyzing the hydrolysis of a phosphodiester bond in RNA. In some embodiments, an endoribonuclease can be a site-directed polypeptide. An endoribonuclease may be a member of a CRISPR system (e.g., Type I, Type II, Type III). Endoribonuclease can refer to a Repeat Associated Mysterious Protein (RAMP) superfamily of proteins (e.g., Cas6, Cas6, Cas5 families). Endoribonucleases can also include RNase A, RNase H, RNase I, RNase III family members (e.g., Drosha, Dicer, RNase N), RNase L, RNase P, RNase PhyM, RNase T1, RNase T2, RNase U2, RNase V1, RNase V.

The term “inhibiting” refers to the ability of a chemical structure to partially or completely inhibit a chemical reaction. A skilled artisan would understand that whether the inhibition is partial or complete depends on the sensitivity of detection methods. The term “inhibiting cleavage” with respect to a nuclease refers to the ability to detectably diminish the amount of cleavage product. The term “preventing cleavage” with respect to a nuclease refers to the ability to diminish the amount of cleavage product below the level of detection.

The term “NATNA” refers to a nucleic acid targeting nucleic acid. NATNA may be a part of the programmable endonuclease system, such as a CRISPR system. NATNA may be comprised of two nucleic acid targeting polynucleotides (“dual guide”) including a CRISPR RNA (crRNA) and transactivating CRISPR RNA (tracrRNA). NATNA may be comprised of an engineered single nucleic acid targeting polynucleotide (“single guide”) comprising crRNA and tracrRNA connected by a fusion region (linker). NATNA may also be comprised of a naturally-occurring single guide (e.g., Cas12a guide RNA). The crRNA may comprise a targeting region and an activating region. The tracrRNA may comprise a region capable of hybridizing to the activating region of the crRNA. The term “targeting region” refers to a region that is capable of hybridizing to a sequence in a target nucleic acid. The term “activating region” refers to a region that interacts with a polypeptide, e.g., a CRISPR nuclease.

Nucleic acids labeled with a donor fluorophore and an acceptor fluorophore (or a reporter fluorophore and a quencher) are a popular type of probe or enzymatic substrate. Nucleic acids labeled with a fluorophore are a popular type of probe or enzymatic substrate. Especially popular are probes and substrates labeled with two fluorophores forming a FRET pair. Popular types of dual-labeled probes include Taqman and Molecular Beacon probes.

In the Tagman assay (U.S. Pat. No. 5,210,015), a dual-labeled oligonucleotide probe hybridizes to the nascent amplification product during PCR. Fluorescence is detected when the 5′-3′ exonuclease activity of the DNA polymerase hydrolyzes the probe between the two fluorophores.

Molecular Beacons are hairpin-shaped dual-labeled probes where binding of a probe to its target causes unraveling of the hairpin. The unraveling separates the FRET pair allowing fluorescence of the donor fluorophore to be detected. Tyagi S, et al., (1996) Molecular beacons: probes that fluoresce upon hybridization. Nat Biotechnol. 14(3):303.

Nucleic acid substrates labeled with two fluorophores forming a FRET pair and capable of conditional fluorescence can be used to detect in vitro enzymatic activity. Enzymatic activity may be indicative for example, of the presence of an infectious agent in the sample. The use of probes labeled with a fluorophore and a quencher fluorophore to detect microbial contamination as described in U.S. Pat. No. 10,663,459. According to this method, the presence of a microorganism in the sample results in cleavage of the probe with one or more nucleases (e.g., endonucleases or exonucleases). All possible cleavages result in physical separation of the fluorophore and the quencher, and emission of detectable fluorescence which is indicative of the presence of a microorganism in the sample.

A similar principle is employed in the U.S. Pat. No. 10,653,800 where an RNA substrate incorporates 2′-O-methyl-modified pyrimidines and is uniquely sensitive to Mycoplasmal RNase. The substrate is labeled with a fluorophore and a quencher. The presence of Mycoplasma in the sample results in digestion of the probe with the Mycoplasmal RNase and emission of detectable fluorescence which is indicative of the presence of Mycoplasma in the sample.

Double-stranded nucleic acid substrates labeled with a fluorophore and a quencher have also been used to detect specific editing activity of CRISPR endonucleases. For example, Smith et al., (2020) Probing CRISPR-Cas12a Nuclease Activity Using Double-Stranded DNA-Templated Fluorescent Substrates, Biochemistry 59:1474, describe cleavage of such a substrate with the trans-cleavage (“trans-shredding”) activity of the CRISPR Cas12a nuclease. The trans-shredding activity is triggered by binding of the Cas12a-crRNA complex to a double-stranded DNA target. The trans-shredding nuclease activity is directed to any double-stranded DNA in the vicinity of the Cas12a-crRNA-target complex. The shredding of the substrate labeled with a fluorophore and a quencher result in emission of detectable fluorescence which is indicative of the formation of the Cas12a-crRNA-target complex in the sample.

Activity of CRISPR-Cas nucleases is typically measured by incubation of the ribonucleoprotein complex (RNP) with a model substrate followed by agarose gel electrophoresis to resolve the cleaved fragments from the intact substrate. This approach is low-throughput and is generally limited to end-point analysis, and thus provides little or no information about the kinetics of the reactions.

A fluorescence-based assay has also been described for Cas12a, however the assay measures only the non-specific trans activity of the Cas12a enzyme. (see Smith C W, Biochemistry. 2020 supra). When the Cas12a RNP engages and cleaves the target sequence (cis activity) the Cas12a is activated to non-specifically degrade short fragments of DNA (trans activity or “trans shredding” activity). There are several limitations to measuring trans activity as an indicator of cis activity. First, the exact correlation between the cis and trans activity is not known. Second, the surrogate assay provides limited kinetic information about the cis activity because the two reactions are performed by the same enzyme and cannot be decoupled. Lastly, the assay lacks general applicability as many endonucleases to not exhibit trans activity.

A fluorescence assay for measuring Cas9 activity was developed in 2018 (see Seamon et al., (2018), Versatile High-Throughput Fluorescence Assay for Monitoring Cas9 Activity, Anal. Chem., 2018, 90, 11, 6913-6921.) However, the assay only provides endpoint analysis and not real-time data because it relies on denaturing the DNA substrate after endonuclease cleavage.

The instant invention overcomes these drawbacks by providing a convenient real-time assay for endonuclease activity. The invention comprises a simple, high throughput fluorescence-based assay as an assessment and quality control tool.

The invention further comprises a diagnostic assay for the specific activity of an endonuclease which is indicative of the presence of a diagnostic target in the sample.

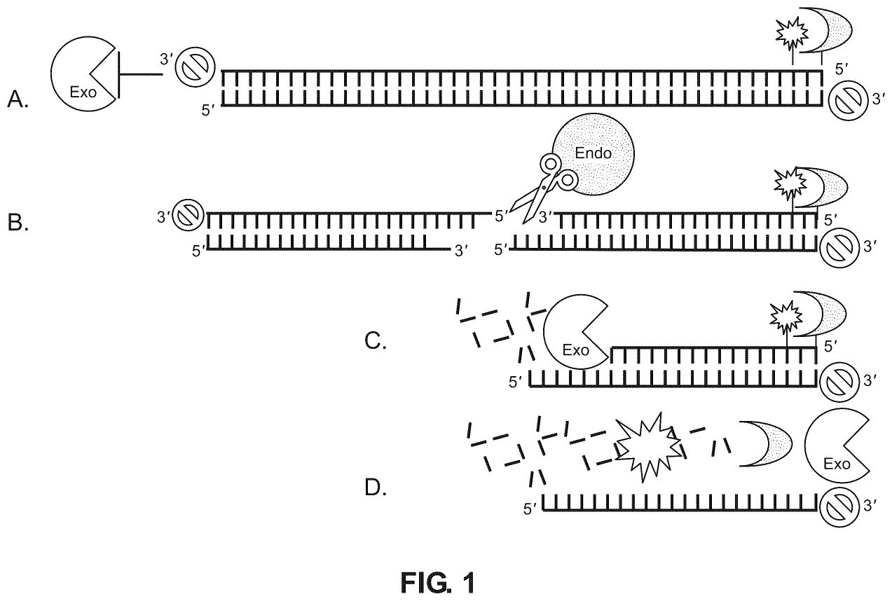

In some embodiments, the invention is a substrate molecule for detecting and assessing the activity of an endonuclease. As shown in FIG. 1, panel A, in some embodiments, the substrate is a nucleic acid having at least one double stranded region. In some embodiments, the substrate is single stranded. In some embodiments, the substrate is a double-stranded nucleic acid comprising two nucleic acid strands forming a duplex via hybridization. In other embodiments, the substrate is a single nucleic acid strand forming a secondary structure comprising a double-stranded region, e.g., a hairpin, via hybridization. One of skill in the art would recognize that hybridization and formation of a double-stranded region do not require 100% complementarity through the length of the nucleic acid strands. Under suitable conditions defined by ionic power of the buffer and temperature, a stable hybrid (a double stranded region) can form with less than 100%, e.g., 90%, 80%, 75% or less complementarity between the two nucleic acid strands.

As is further seen on FIG. 1, the nucleic acid substrate comprises a donor fluorophore and an acceptor fluorophore (or a reporter fluorophore and a quencher fluorophore), said fluorophores forming a Fluorescence Resonance Energy Transfer (FRET) pair.

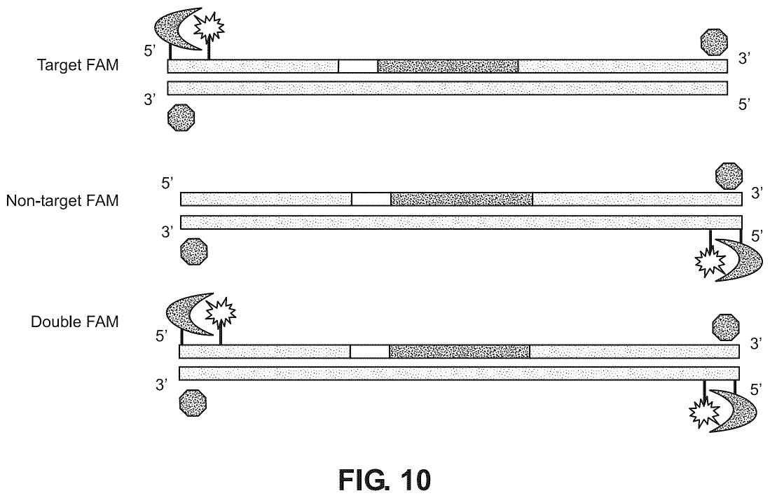

The fluorophore and the quencher may be placed in various locations on the double-stranded nucleic acid substrate. Additionally, more than one fluorophore may be used. As shown in FIG. 10, the fluorophore and the quencher may be placed on the target strand, on the non-target strand, and on both target and non-target strands.

Fluorescence resonance energy transfer (FRET), also known as Foerster (or Forster) resonance energy transfer is transfer of excitation energy from one molecule to another without fluorescence and re-absorption. A donor chromophore enters an electronically excited state after having absorbed light of a certain wavelength. The donor transfers energy to an acceptor and the acceptor is promoted to its electronically excited state. Subsequently, the electronically excited state of the acceptor decays so that in turn detectable light is emitted. Because the acceptor diminishes or quenches fluorescence of the donor, the acceptor is sometimes referred to as a quencher. The donor is sometimes referred to as a reporter. In conventional FRET technology donor and acceptor are both fluorophores. The donor fluorophore absorbs the light of a certain absorption wavelength and the acceptor emitted light of a particular emission wavelength which is longer than the absorption wavelength. FRET occurs when donor and acceptor are in close proximity (e.g., 1-10 nm). In some embodiments, the donor fluorophore and the acceptor fluorophore are placed between 0 and 12 nucleotides apart. The donor fluorophore and the acceptor fluorophore are placed on the same strand of the substrate or on different (opposite) strands of the substrate. Either the donor, the acceptor or both the donor and the acceptor can be placed near a terminus of a nucleic acid strand, e.g., a 5′-terminus or a 3′-terminus. The donor and the acceptor fluorophores may be on the same strand or on opposite strands. A skilled practitioner would recognize various options for placing the donor fluorophore and the acceptor fluorophore (or the reporter fluorophore and the quencher) within the double-stranded substrate so that the desired proximity of the fluorophores (e.g., 1-10 nm) is achieved.

Existing literature provides ample guidance for selecting appropriate reporter-quencher pairs capable of FRET, see e.g., U.S. Pat. Nos. 5,538,848; 8,350,038; and 8,137,616. Generally, as recommended in U.S. Patent Application Publication US20060088855, the donor fluorophores absorb in the range of 350-800 nm, preferably 350-600 nm or 500-750 nm and the distance between donor and acceptor be 10 to 100 angstroms. See also Pesce et al., eds., Fluorescence Spectroscopy (Marcel Dekker, New York, 1971); White et al., Fluorescence Analysis: A Practical Approach (Marcel Dekker, New York, 1970); Berlman, Handbook of Fluorescence Spectra of Aromatic Molecules, 2nd Edition (Academic Press, New York, 1971); Griffiths, Color and Constitution of Organic Molecules (Academic Press, New York, 1976); Bishop, editor, Indicators (Pergamon Press, Oxford, 1972); Haugland, Handbook of Fluorescent Probes and Research Chemicals (Molecular Probes, Eugene, 1992); Pringsheim, Fluorescence and Phosphorescence (Interscience Publishers, New York, 1949); and the like.

Many donors, acceptors and donor/acceptor pairs that exhibit FRET phenomenon are commercially available. Popular donors include fluorescein dyes such as 5-carboxyfluorescein (5-FAM), 6-carboxyfluorescein (6-FAM), 2′,4′,1,4,-tetrachlorofluorescein (TET), 2′,4′,5′,7′, 1,4-hexachlorofluorescein (HEX), and 2′,7′-dimethoxy-4′,5′-dichloro-6-carboxyfluorescein (JOE). Other donors include coumarin dyes, Alexa Fluor family of dyes, IRDye 800CW, Cascade Blue, Pacific Blue, Pacific Orange, and Texas Red. Popular acceptors include rhodamine dyes such as tetramethyl-6-carboxyrhodamine (TAMRA), and tetrapropano-6-carboxyrhodamine (ROX), DABSYL, DABCYL, cyanine dyes including Cy5 and Cy5.5, anthraquinone, nitrothiazole, and nitroimidazole compounds. Additional acceptors are LC-Red 610, LC-Red 640, LC-Red 705, JA286, DDQ-I, DDQ-II, QSY-7, QSY-21, IRDye QC1, Iowa Black FQ and Iowa Black RQ. and sulfonated cyanine dyes disclosed in U.S. Pat. No. 6,027,709. Popular donor-acceptor combinations include fluorescein/rhodamine, especially carboxyfluorescein/tetramethyl-rhodamine (FAM/TAMRA). TAMRA as quencher can also be paired with such donors as HEX (hexachloro-fluorescein), TET (tetrachloro-fluorescein), JOE (5′-Dichloro-dimethoxy-fluorescein) and cyanine dyes. Another donor/acceptor pair is disclosed in the U.S. Pat. No. 9,796,746 and is composed of an oxidized form of a carbaNADH-based first fluorophore and a second fluorophore that is excitable with light having a wavelength of 445-540 nm and an emission maximum greater than 560 nm.

Another group of fluorescent compounds are the naphthylamines, having an amino group in the alpha or beta position. Included among such naphthylamino compounds are 1-dimethyl-aminonaphthyl-5-sulfonate, 1-anilino-8-naphthalene sulfonate and 2-p-touidinyl-6-naphthalene sulfonate. Other dyes include 3-phenyl-7-isocyanatocoumarin, acridines, such as 9-isothiocyanatoacridine and acridine orange; N-(p-(2-benzoxazolyl)phenyl)maleimide; benzoxadiazoles, stilbenes, and pyrenes.

Another category of fluorophores are the BODIPY® dyes described in U.S. Pat. No. 5,994,063. “BODIPY®” refers to a class of modified, spectrally-discriminating fluorophores wherein the parent heterocyclic molecule is a dipyrrometheneboron difluoride compound. Most BODIPY® fluorophores have adsorption maxima of about 450 to 700, and emission maxima of about 450 to 700. Examples include BODIPY® 503/512-SE (4,4-difluoro-5,7-dimethyl-4-bora-3a,4a-diaza-s-indacene-3-propionicacid), BODIPY®523/547 (4,4-difluoro-5-phenyl-4-bora-3a,4a-diaza-s-indacene-3-propionic acid), BODIPY® 530/550 (4,4-difluoro-5,7-diphenyl-4-bora-3a,4a-diaza-s-indacene-3-propionic acid), BODIPY® 558/568 (4,4-difluoro-5-(2-thienyl)-4bora-3a,4a-diaza-s-indacene-3-propionic acid), BODIPY® 564/570 (4,4-difluoro-5-styryl-4-bora-3a,4a-diaza-s-indacene-3-propionicacid), BODIPY® 576/589 (4,4-difluoro-5-(2-pyrrolyl)-4-bora-3a,4a-diaza-s-indacene-3-propionic acid), and BODIPY® 581/591 (4,4-difluoro-5-(4-phenyl-1,3-butadienyl)-4-bora-3a,4a-diaza-s-indacene-3-propionic acid).

One type of quenchers are “dark quenchers.” These non-fluorescent acceptors enable low background fluorescence and thus improve assay sensitivity. When a dark quencher is used, the donor fluorophore does not emit light until the quencher is removed from the proximity of the donor. For example, if donor and quencher are conjugated to an oligonucleotide, fluorescence of the donor may occur only when the quencher is removed though hydrolysis of the oligonucleotide by a nuclease. One example of a dark quencher is DABCYL (4-[[4-(dimethylamino)-phenyl]-azo]-benzoic acid) which quenches donor dyes in a range of from 380 to 530 nm. Another dark quencher is Eclipse Quencher (4-[[2-chloro-4-nitro-phenyl]-azo]-aniline (available from Epoch Biosciences, Inc.) which has an absorption maximum at 530 nm and efficiently quenches over a spectrum from 520 to 670 nm. Yet another category of dark quenchers is the Black Hole Quenchers, such as BHQ-1 ([(4-(2-nitro-4-methyl-phenyl)-azo)-yl-((2-methoxy-5-methyl-phenyl)-azo)]-aniline) and BHQ-2 ([(4-(1-nitro-phenyl)-azo)-yl-((2,5-dimethoxy-phenyl)-azo)]-aniline) (all available from Biosearch Technologies, Inc.).

Another type of quencher includes the pyridinyl-isoquinoline-dione derivatives disclosed in the U.S. Pat. No. 8,350,038. These compounds feature a low background signal and high quenching efficiency. Yet another category of quenchers is the non-fluorescent cyanine quencher compounds attached to base of a nucleotide via a linker compound disclosed in the U.S. Pat. No. 6,348,596. Yet another category of quenchers is the weakly luminescent cyanines that are substituted by one or more heteroaromatic quenching moieties disclosed in the U.S. Pat. No. 8,093,411. These quenchers exhibit little or no observable luminescence and efficiently quench a broad spectrum of luminescent compounds.

Methods of synthesizing oligonucleotides and of covalently attaching fluorophores to nucleic acids are known in the art. See e.g., U.S. Pat. Nos. 3,996,345; 4,351,760; 4,757,141; 4,739,044; 4,997,928; 5,538,848; 5,188,934; 5,231,191; and 7,759,469; and Eckstein, ed., Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19:3019 (1991) (3′ sulfhydryl); Giusti et al., PCR Methods and Applications, 2:223-227 (1993); Agrawal et al., Tetrahedron Letters, 31:1543-1546 (1990) (attachment via phosphoramidate linkages); Sproat et al., Nucleic Acids Research, 15:4837 (1987) (5′ mercapto group); Nelson et al., Nucleic Acids Research, 17:7187-7194 (1989) (3′ amino group). For synthesizing labeled nucleic acid probes, functional groups and linking moieties may be used. As commonly used, pre-synthesized fluorophore-labeled nucleotides are incorporated into an oligonucleotide using standard phosphoramidite-based chemistry. By incorporating such nucleotides into a desired position in an oligonucleotide, donor and acceptor fluorophores may be incorporated at any internal or terminal position in the oligonucleotide. In the pre-synthesized fluorophore-labeled nucleotides the label may be bound by a functional group attached for example, to an amino group of a nucleotide's base. In other embodiments, the label is attached to a part of a nucleotide via a linking moiety. For example, in some embodiments, the nucleotide base is modified to allow conjugation to a label. For example, U.S. Pat. No. 7,759,469 discloses substituted nitroindole nucleotides that can be conjugated to a fluorophore.

In some embodiments, the double-stranded nucleic acid substrate is between about 10 and about 90 base pairs in length. For example, for endonuclease Cas12a, the substrate is between 35 and 90 base pairs long and may comprise PAM (5 nt), a spacer (20 nt), end protection (5 nt on each end), adding to 35 base pairs. In general, endonucleases have different sizes and structures of recognition sequences. Therefore, for each endonuclease to be tested an optimal length of substrate may be determined using the calculation shown above. The optimal length and sequence can be determined in silico or found empirically. Such optimal length enables the most efficient digestion by the endonuclease without steric hindrance, while not having an excessive length. The excessive length is associated with excessive cost of manufacture and requires additional units of exonuclease and additional time to perform the method described herein. An optimal length of the double-stranded nucleic acid substrate for each endonuclease to be tested may incorporate one or more of such considerations.

In some embodiments, the nucleic acid substrate of the invention includes chemical modifications. In some embodiments, the modification effects increased stability of the nucleic acid duplex. In some embodiments, the modification confers resistance to nuclease digestion or inhibition of nucleases. In some embodiments, the modification confers resistance to exonuclease digestion or inhibition of exonucleases.

In some embodiments, the modification is a backbone modification. One type of backbone modification is a modified internucleoside linkage. For example, the modification includes phosphorothioate linkages and heteroatom internucleoside linkages.

Another type of backbone modification is modification of a sugar moiety. In some embodiments, the modification involves incorporation of a 6-membered morpholino ring in place of a ribose or deoxyribose ring. Another backbone modification involves incorporation of a cyclohexenyl ring in place of a ribose or deoxyribose (ceNA). Yet another backbone modification involves incorporation of Locked Nucleic Acids (LNAs) in which the 2′-hydroxyl group is linked to the 4′ carbon atom of the ribose thereby forming a bicyclic structure having a 2′-C,4′-C-oxymethylene linkage. LNAs are characterized by duplex stability and resistance to 3′-5′ exonuclease digestion.

In some embodiments, the modification is a nitrogenous base modification. For example, the double-stranded nucleic substrate may incorporate one or more 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (Hpyrido(3′,2′:4,5)pyrrolo(2,3-d)pyrimidin-2-one), 7-deaza-adenine, 7-deazaguanosine, 2-aminopyridine and 2-pyridone. Nucleobases can be useful for increasing the binding affinity of a polynucleotide compound. These can include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

One of skill in the art will appreciate that nucleic acid modifications should not be included in the recognition site for the endonuclease to be tested if the modification may interfere with the endonuclease activity, unless it has been established that the endonuclease is not inhibited by the modification. Similarly, one of skill in the art will appreciate that nucleic acid modifications known to inhibit exonuclease digestion should not be included in portion of the substrate located between the endonuclease recognition site and the fluorophore so as not to block or inhibit the performance of the method described herein.

The methods and compositions described herein utilize an exonuclease. Exonucleases are known in the art, and many are commercially available, see e.g., Lovett S. T. (2011). The DNA Exonucleases of Escherichia coli. EcoSal Plus, 4(2) and Shevelev, I., Hübscher, U. (2002) The 3′-5′ exonucleases. Nat Rev Mol Cell Biol 3, 364-376. Many exonucleases are available from New England Biolabs (Ipswich, Mass.). For example, Exonuclease I, Exonuclease T, and Exonuclease VII are 3′-5′ exonucleases active on single-stranded DNA. RecJf is a 5′-3′ exonuclease active on single-stranded DNA. Exonuclease III is a 3′-5′ exonuclease active on single-stranded DNA and double-stranded DNA. T7 Exonuclease, Exonuclease V, Exonuclease VIII, Lambda Exonuclease, and T5 Exonuclease are 5′-3′ exonucleases active on single-stranded DNA and double-stranded DNA. Exonuclease V (RecBCD) and BAL-31 are simultaneously 5′-3′ and 3′-5′ exonucleases active on single-stranded DNA and double-stranded DNA. Depending on the type of nucleic acid termini generated by the action of the endonuclease to be tested, a skilled practitioner is able to select an appropriate exonuclease. The appropriate exonuclease would utilize the terminus or termini generated by the endonuclease and hydrolyze the nucleic acid substrate described herein. The hydrolysis by exonuclease will separate the FRET pair of fluorophores (e.g., the donor and the acceptor or the reporter and the quencher) so that fluorescence or a change in fluorescence could be detected.

As shown in FIG. 1, the substrate comprises a structure at at least one of the termini that inhibits or prevents digestion by an exonuclease. The choice of an exonuclease to be included in the methods, compositions and kits described herein informs the choice of the exonuclease inhibiting structure conferring end protection in the nucleic acid substrate. For example, if the preferred substrate for the exonuclease is the 3′-end, the nucleic acid substrate has an exonuclease inhibiting structure at each of the 3′-ends. If the preferred substrate for the exonuclease is the 5′-end, the nucleic acid substrate has an exonuclease inhibiting structure at each of the 5′-ends. If the exonuclease is active at both 3′-ends and 5′-ends, the nucleic acid substrate has an exonuclease inhibiting structure at each of the 3′-ends and 5′-ends.

The choice of the exonuclease must also agree with the types of breaks and available ends generated by the endonuclease to be tested. The exonuclease (or a mixture of exonucleases) must be active on the types of ends (protruding, blunt or recessed, hydroxyl or phosphoryl) generated by the endonuclease to be tested, while being inhibited by the inhibitory structures present at the ends of the nucleic acid substrate described herein.

In some embodiments, the endonuclease is a nickase, i.e., an endonuclease cleaving only one strand of the duplex and generating a nick. In this embodiment, the exonuclease may be for example, Exonuclease III, T5 Exonuclease, T7 Exonuclease, Lambda Exonuclease, and BAL31 Exonuclease.

In some embodiments, the endonuclease cleaves both strands of the duplex and generates a double-stranded break. In some embodiments, the double-stranded break has blunt ends. In some embodiments, the double-stranded break has staggered ends. In some embodiments, staggered ends may have a protruding 3′-end. In some embodiments, the staggered ends may have a protruding 5′-end. A skilled practitioner would be able to select an exonuclease (or a mixture of two or more exonucleases) capable of initiating hydrolysis in the desired direction from the particular type of ends generated by the endonuclease to be tested. For example, New England Biolabs, Inc. (Ipswich, Mass) publicizes a list of available exonucleases grouped by biochemical properties (e.g., type of end required and directionality of hydrolysis).

As is further seen in FIG. 1, panel B, the substrate comprises a recognition sequence (or a recognition site) for an endonuclease. Depending on the endonuclease to be tested, the recognition site may comprise a cleavage site and have one or more additional elements. In some embodiments, the endonuclease to be tested is a nucleic acid-guided endonuclease. In some embodiments, the endonuclease to be tested is a CRISPR Class I (CASCADE) endonuclease or a CRISPR Class II endonuclease. In some embodiments, the endonuclease to be tested is a CRISPR Cas9 or CRISPR Cas12a (Cpf1) endonuclease.

In some embodiments, the endonuclease is a deoxyribonuclease and the substrate is single stranded or double-stranded DNA. In some embodiments, the endonuclease is a ribonuclease and the substrate is single stranded or double-stranded RNA. In some embodiments, the endonuclease is a ribonuclease such as a ribozyme, a hammerhead ribozyme, a DNAzyme, a PNAzyme or an engineered endoribonuclease e.g., of the type described in Choudhury, R. et al., (2012) Engineering RNA endonucleases with customized sequence specificities. Nature Comm., 3:1147.

In some embodiments, the endonuclease to be tested is or is related to an endonuclease encoded by the CRISPR locus. The CRISPR (Clustered Regularly Interspaced Short Palindromic Repeats) genomic locus is found many prokaryotic genomes and provides resistance to invasion of foreign nucleic acids. Structure, nomenclature and classification of CRISPR loci are reviewed in Makarova et al., Evolution and classification of the CRISPR-Cas systems. Nature Reviews Microbiology. 2011 June; 9(6): 467-477.

Briefly, a typical CRISPR locus includes a number of short repeats regularly interspaced with spacers. The CRISPR locus also includes coding sequences for CRISPR-associated (Cas) genes. A spacer-repeat sequence unit encodes a crisprRNA (crRNA). In vivo, a mature crRNAs is processed from a polycistronic transcript referred to as pre-crRNA or pre-crRNA array. The repeats in the pre-crRNA array are recognized by Cas-encoded proteins that bind to and cleave the repeats liberating mature crRNAs. CRISPR systems perform cleavage of a target nucleic acid wherein Cas proteins and crRNA form a CRISPR ribonucleoproteins (crRNP). The crRNA molecule guides the crRNP to the target nucleic acid (e.g., a foreign nucleic acid invading a bacterial cell) and the Cas nuclease proteins cleave the target nucleic acid.

Class 1, Type I CRISPR systems include means for processing the pre-crRNA array that include a multi-protein complex called Cascade (CRISPR-associated complex for antiviral defense) comprised of subunits CasA, B, C, D and E. The Cascade-crRNA complex recognizes the target nucleic acid through hybridization of the target nucleic acid with crRNA. The bound nucleoprotein complex recruits the Cas3 helicase/nuclease to facilitate cleavage of target nucleic acid.

Class 2, Type II CRISPR systems include a trans-activating CRISPR RNA (tracrRNA). The tracrRNA hybridizes to a crRNA repeat in the pre-crRNA array and recruits endogenous RNaseIII to cleave the pre-crRNA array. The tracrRNA/crRNA complex can associate with a nuclease, e.g., Cas9. The crRNA-tracrRNA-Cas9 complex recognizes the target nucleic acid through hybridization of the target nucleic acid with crRNA. Hybridization of the crRNA to the target nucleic acid activates the Cas9 nuclease. for target nucleic acid cleavage.

Class 1, Type III CRISPR systems include the RAMP superfamily of endoribonucleases (e.g., Cas6) that cleave the pre-crRNA array with the help of one or more CRISPR polymerase-like proteins.

Class 2, Type V CRISPR systems comprise a different set of Cas-like genes, including Csf1, Csf2, Csf3 and Csf4 which are distant homologues of Cas genes in Type I-III CRISPR systems.

As shown in FIG. 1, panel B, the substrate comprises a recognition sequence (or a recognition site) for an endonuclease. In some embodiments, the recognition sequence and the cleavage site are the same. In some embodiments, the recognition site is a palindromic sequence characteristic of Type II restriction endonucleases, and the cleavage occurs within the palindromic sequence. In some embodiments, the recognition sequence is distinct from the cleavage site. In some embodiments, the endonuclease is a CRSIPR Cas9 endonuclease, and the recognition sequence is the protospacer adjacent motif (PAM), while the cleavage site is adjacent to the PAM. In some embodiments, the endonuclease is a CRSIPR Cas12a endonuclease, and the recognition sequence is the protospacer adjacent motif (PAM), while the cleavage site is 16-18 bases away from the PAM on the non-target strand, and 23-25 bases away from the PAM on the target strand, see Strohkendl, I., et al., (2018) Kinetic Basis for DNA Target Specificity of CRISPR-Cas12a, Mol. Cell, 71(5):816-824.e3.

In some embodiments, the endonuclease is a nickase, i.e., an endonuclease cleaving only one strand of the duplex and generating a nick. In some embodiments, the endonuclease cleaves both strands of the duplex and generates a double-stranded break. The double-stranded break may have blunt ends or staggered ends. The staggered ends may have a protruding 3′-end or a protruding 5′-end. The 5′-end may have a 5′-phosphoryl or a 5′-hydroxyl group, while the 3′-end may have a 3′-phosphoryl or a 3′-hydroxyl group.

CRISPR nucleases do not cleave a fixed sequence but instead are guided by a nucleic acid guide as described above. In addition to the guide RNA, the CRISPR nucleases recognize an additional sequence termed protospacer adjacent motif (PAM). In some embodiments, the substrate of the invention comprises a protospacer adjacent motif (PAM). In the embodiments where the endonuclease to be tested is a CRISPR Class I (CASCADE) endonuclease, the substrate includes a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In the embodiments where the endonuclease to be tested is a CRISPR Class II endonuclease, the substrate includes a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, and 5′-NNNACA-3′, 5′-TTN-3′, 5′-TTTN-3′ and 5′-TTTV-3′.

CRISPR nucleases do not cleave a fixed recognition sequence but instead are guided by a nucleic acid guide termed “guide RNA” and termed herein “a nucleic acid targeting nucleic acid (NATNA).” The guide RNA (NATNA) comprises a “spacer” sequence complementary to the endonuclease cleavage site. In the embodiments where the endonuclease to be tested is a CRISPR endonuclease, the substrate includes a target sequence capable of hybridizing to a portion (“spacer”) in NATNA.

In some embodiments, the endonuclease is a nucleic acid guided endonuclease. The reaction mixture with such an endonuclease further requires a nucleic acid targeting nucleic acid (NATNA). In some embodiments, the endonuclease is a CRISPR endonuclease and the NATNA is guide RNA. The endonuclease is capable of forming a ribonucleoprotein complex (RNP) with one or more guide RNAs. In some embodiments, the endonuclease is a Class 2, Type II CRISPR endonuclease and NATNA comprises tracrRNA and crRNA. In some embodiments, the endonuclease is a Class 2, Type V CRISPR endonuclease and NATNA comprises crRNA.

In some embodiments, the NATNA is selected from the embodiments described in U.S. Pat. No. 9,260,752. Briefly, a NATNA can comprise, in the order of 5′ to 3′, a spacer extension, a spacer, a minimum CRISPR repeat, a single guide connector, a minimum tracrRNA, a 3′ tracrRNA sequence, and a tracrRNA extension. In some instances, a nucleic acid-targeting nucleic acid can comprise, a tracrRNA extension, a 3′ tracrRNA sequence, a minimum tracrRNA, a single guide connector, a minimum CRISPR repeat, a spacer, and a spacer extension in any order.

In some embodiments, the guide nucleic acid-targeting nucleic acid can comprise a single guide NATNA. The NATNA comprises a spacer sequence which can be engineered to hybridize to the target nucleic acid sequence. The NATNA further comprises a CRISPR repeat comprising a sequence that can hybridize to a tracrRNA sequence. Optionally, NATNA can have a spacer extension and a tracrRNA extension. These elements can include elements that can contribute to stability of NATNA. The CRISPR repeat and the tracrRNA sequence can interact, to form a base-paired, double-stranded structure. The structure can facilitate binding of the endonuclease to the NATNA.

In some embodiments, the single guide NATNA comprises a spacer sequence located 5′ of a first duplex which comprises a region of hybridization between a minimum CRISPR repeat and minimum tracrRNA sequence. The first duplex can be interrupted by a bulge. The bulge facilitates recruitment of the endonuclease to the NATNA. The bulge can be followed by a first stem comprising a linker connecting the minimum CRISPR repeat and the minimum tracrRNA sequence. The last paired nucleotide at the 3′ end of the first duplex can be connected to a second linker connecting the first duplex to a mid-tracrRNA. The mid-tracrRNA can comprise one or more additional hairpins.

In some embodiments, the NATNA can comprise a double guide nucleic acid structure. The double guide NATNA comprises a spacer extension, a spacer, a minimum CRISPR repeat, a minimum tracrRNA sequence, a 3′ tracrRNA sequence, and a tracrRNA extension. The double guide NATNA does not include the single guide connector. Instead, the minimum CRISPR repeat sequence comprises a 3′ CRISPR repeat sequence and the minimum tracrRNA sequence comprises a 5′ tracrRNA sequence and the double guide NATNAs can hybridize via the minimum CRISPR repeat and the minimum tracrRNA sequence.

In some embodiments, the NATNA is an engineered guide RNA comprising one or more DNA residues (CRISPR hybrid RDNA or chRDNA). In some embodiments, NATNA is selected from the embodiments described in U.S. Pat. No. 9,650,617. Briefly, some chRDNA for use with a Class 2 CRISPR system may be composed of two strands forming a secondary structure that includes an activating region composed of an upper duplex region, a lower duplex region, a bulge, a targeting region, a nexus, and one or more hairpins. A nucleotide sequence immediately downstream of a targeting region may comprise various proportions of DNA and RNA. Other chRDNA may be a single guide D(R)NA for use with a Type II CRISPR system comprising a targeting region, and an activating region composed of and a lower duplex region, an upper duplex region, a fusion region, a bulge, a nexus, and one or more hairpins. A nucleotide sequence immediately downstream of a targeting region may comprise various proportions of DNA and RNA. For example, the targeting region may comprise DNA or a mixture of DNA and RNA, and an activating region may comprise RNA or a mixture of DNA and RNA.

In some embodiments, the guide RNA includes nucleic acid modifications, e.g., the modifications conferring resistance to ribonucleases. This feature is especially advantageous in crude lysate assays described below.

In some embodiments, the endonuclease to be tested is a restriction endonuclease. In some embodiments, the endonuclease to be tested is a Type II, II or IV restriction endonuclease. For each endonuclease, the substrate of the invention contains the appropriate recognition sequence. For the Type IV restriction endonuclease, the substrate of the invention also contains one or more methylated residues needed for cleavage by the endonuclease.

In embodiments where the endonuclease to be tested is a zinc finger nuclease (ZFN), or a ZFN conjugated to the non-specific cleavage domain of the restriction endonuclease Fok I, the target sequence is about 22-52 bases long and comprises a pair of ZFN recognition sequences, each 9-18 nucleotides long, separated by a spacer, which is 4-18 nucleotides long. (see e.g., Kim Y. G., et al., (1996). Hybrid restriction enzymes: zinc finger fusions to Fok I cleavage domain, Proc Natl Acad Sci USA. 93(3): 1156-1160.

In the embodiments where the endonuclease to be tested is a transcription activator-like effector nuclease (TALEN), or a TALEN-Fok I fusion, the target sequence is about 48-85 nucleotides long and comprises a pair of TALEN recognition sequences, each 18-30 bases long, separated by a spacer, which is 12-25 bases long. (see e.g., Christian M. et al., (2010) Targeting DNA double-strand breaks with TAL effector nucleases, Genetics. 186 (2): 757-61.

In some embodiments, the endonuclease to be tested is an Argonaute (Ago) endonuclease. Ago endonucleases do not have a recognition sequence but are guided by small interfering DNA guides (siDNA) to cleave complementary DNA. Hegge, et al., (2019) DNA-guided DNA cleavage at moderate temperatures by Clostridium butyricum Argonaute, N.A. R. 47(11):5809.

In some embodiments, the endonuclease to be tested is an Arcus endonuclease. Arcus is a I-CreI endonuclease with a 22 bases long target sequence (see e.g., Durrenberger et al., (1991) Double-strand break induced recombination in Chlamydomonas reinhardtii chloroplasts, N.A.R. 24(17):3323.

In some embodiments, the endonuclease to be tested is an endoribonuclease and the substrate comprises RNA. In some embodiments, the substrate is a single-stranded RNA. In some embodiments, the substrate is a double-stranded RNA. In some embodiments, the substrate is an RNA-DNA hybrid. The exonuclease used in such an assay is an exodeoxyribonuclease or exoribonuclease to accommodate the chosen substrate. Examples of endoribonucleases cleaving one or more of such substrates include RNase III, RNase A, RNase T1, RNase A, RNase H, RNase Z and RNase P. Other examples of endoribonucleases are CRISPR endoribonucleases selected from Cas13 and Cas7-11.

Examples of exoribonucleases include those cleaving in the 3′-5′ direction such as RNase R, RNase II, RNase D, RNase T, RNase BN, RNase PH, and those cleaving in the 5′-3′ direction such as Exoribonuclease I, and Exoribonuclease II.

In some embodiments, the invention is a method for detecting activity of an endonuclease using the substrate described herein. As shown in FIG. 1, panel B, the method comprises contacting an endonuclease to be tested with a reaction mixture including a double-stranded nucleic acid substrate comprising: a donor fluorophore and an acceptor fluorophore (or a reporter fluorophore and a quencher fluorophore), the fluorophores forming a Fluorescence Resonance Energy Transfer (FRET) pair and a recognition sequence for the endonuclease to be tested. The reaction mixture is incubated under the conditions suitable for the endonuclease cleavage of its recognition sequence.

The method further comprises contacting the reaction mixture with an exonuclease. The exonuclease, the substrate and the endonuclease can be added simultaneously or consecutively in any order.

As shown in FIG. 1, panels B-C, the structure of the double-stranded nucleic acid substrate is such that the exonuclease does not hydrolyze the double-stranded nucleic acid substrate until endonuclease cleavage takes place. The relevant ends of the double-stranded nucleic acid substrate comprise structures inhibiting cleavage of the substrate by the exonuclease. As described herein, depending on the ends generated by the endonuclease, the exonuclease is a 3′-5′ exonuclease and the 3′-end of the substrate is protected by the exonuclease-inhibiting structure. In other embodiments, the exonuclease is a 5′-3′ exonuclease and the 5′-end of the substrate is protected by the exonuclease-inhibiting structure. In some embodiments, a mixture of a 3′-5′ exonuclease and a 5′-3′ exonuclease is used. In such embodiments, both the 3′-end and the 5′-end of the substrate are protected by the exonuclease-inhibiting structure.

As shown in FIG. 1, panel C, the reaction mixture is further incubated under the conditions suitable for exonuclease cleavage of the double-stranded nucleic acid substrate. In some embodiments, the conditions for endonuclease cleavage and the subsequent exonuclease cleavage are the same. In such embodiments, no change in buffers or incubation conditions are necessary. In some embodiments, no purification steps are performed between contacting the reaction mixture with the endonuclease and contacting the reaction mixture with the exonuclease.

One of the advantages of the method is that it minimizes background signal because the fluorophore and quencher can be placed in very close proximity. It also avoids any steric effects that would likely occur if the fluorophore quencher pair were incorporated near the cut site, for instance with the fluorophore on one side of the cut-site and the quencher on the other.

In some embodiments, the endonuclease to be tested by the method is a nucleic acid-guided endonuclease. In such embodiments, a NATNA is also utilized in the method. In some embodiments, the endonuclease is a CRISPR endonuclease and NATNA is a guide RNA.

In some embodiments, the endonuclease to be tested by the method is a CRISPR Class I (CASCADE) endonuclease, the double-stranded nucleic acid substrate used in the method includes a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In the embodiments where the endonuclease to be tested by the method is a CRISPR Class II endonuclease, the double-stranded nucleic acid substrate used in the method includes a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, and 5′-NNNACA-3′, 5′-TTN-3′, 5′-TTTN-3′ and 5′-TTTV-3′.

In some embodiments, the endonuclease to be tested by the method is one of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In these embodiments, the double-stranded nucleic acid substrate used in the method includes a suitable recognition site.

In some embodiments, the method includes screening, testing, or comparing several endonucleases. In these embodiments, the method comprises contacting a series of reaction mixtures comprising the same ingredients with a series of different endonucleases. In some embodiments, the series of endonucleases is a series of nucleic acid-guided endonucleases and the reaction mixtures in the series of reaction mixtures comprising the same ingredients also comprise the same NATNA. In some embodiments, the series of endonucleases is a series of CRISPR endonucleases and the reaction mixtures in the series of reaction mixtures comprising the same ingredients also comprise the same guide RNA.

In some embodiments, endonuclease is a nucleic acid-guided endonuclease, and the method includes screening, testing, or comparing several NATNAs. In these embodiments, the method comprises contacting a series of reaction mixtures comprising the same ingredients, including the same endonuclease, with a series of different NATNAs. In some embodiments, the endonuclease is a CRISPR endonuclease and the NATNA is a guide RNA. In these embodiments, the method comprises contacting a series of reaction mixtures comprising the same ingredients, including the same CRISPR endonuclease, with a series of different guide RNAs.

In some embodiments, the method includes screening, testing, or comparing several preparations of the same endonuclease. In these embodiments, the method comprises contacting a series of reaction mixtures comprising the same ingredients with a series of different preparations of the same endonuclease. The different preparations may be different isolates of the same endonuclease. The different preparations may be elution aliquots from a chromatography procedure aimed at isolating the endonuclease. The invention comprises a method of monitoring an elution of endonuclease by performing the endonuclease activity assay described herein on emerging elution fractions from a chromatography procedure and retaining the elution fractions with the highest activity of endonuclease.

In some embodiments, the method includes screening, testing, or comparing several nucleic acid sequences in order to identify a preferred or optimal target sequence for an endonuclease. In these embodiments, the method comprises contacting a series of reaction mixtures comprising the same ingredients including the same endonuclease with a series of double-stranded nucleic acid substrates having different sequences. In some embodiments, the endonuclease is a CRISPR endonuclease and the reaction mixtures in the series of reaction mixtures comprising the same ingredients also comprise the same guide RNA.

In some embodiments, the method includes screening, testing, or comparing several reaction conditions in order to identify preferred or optimal reaction conditions for an endonuclease. In some embodiments, the method comprises contacting a series of reaction mixtures comprising different buffer configurations with the same endonuclease. In some embodiments, the series of reaction mixtures is also subjected to different temperature profile during the endonuclease digestion step.

In some embodiments, the method includes screening, testing, or comparing several reaction conditions in order to identify preferred or optimal endonuclease concentrations in nucleic acid cleavage reactions. In some embodiments, the method comprises contacting a series of reaction mixtures identical but for different concentrations of the same endonuclease to be tested.

In some embodiments, the method includes screening, testing, or comparing several CRISPR polynucleotide guides (guide RNAs or gRNAs) in order to identify preferred or optimal gRNA for a CRISPR endonuclease. In these embodiments, the method comprises contacting a series of reaction mixtures identical but for different guide RNAs. In some embodiments, the CRISPR polynucleotide guide comprises one or more DNA residues (CRISPR hybrid RDNA or chRDNA). In these embodiments, the method comprises contacting a series of reaction mixtures identical but for different chRDNAs.

As shown in FIG. 1, panel D, the method next comprises measuring fluorescence emitted by the reaction mixture, wherein a change in fluorescence indicates activity of the endonuclease. The change in fluorescence includes a change in color (wavelength) of the fluorescent signal as well as appearance of fluorescence where fluorescence was previously not detectable. In some embodiments, the measurement is qualitative indicating the presence or absence of endonuclease activity. In other embodiments, the measurement is quantitative indicating the relative amount of endonuclease activity. In some embodiments, the change in fluorescence further includes a change in the intensity of fluorescence and the difference in fluorescence among the samples being tested.

In some embodiments the method described herein is performed with isolated nucleic acid substrates and isolated polypeptides (e.g., endonuclease and exonuclease). In other embodiments, the assay is performed with crude mixtures without substantial purification steps. In some embodiments, an isolated or purified nucleic acid substrate is added to crude isolates or emergent fractions of endonuclease, for example, to rapidly assess the endonuclease production or purification process. In some embodiments, isolated or purified polypeptides (e.g., endonuclease and exonuclease) are added to crude isolates of nucleic acids, for example, minimally treated patient samples to rapidly detect the presence of an infectious agent in the patient.

In some embodiments, the invention is a double stranded nucleic acid substrate and a method of preparing the substrate. The substrate has exonuclease end-protection such as e.g., phosphorothioate bonds. 1, 2, 3, 4, or about 5 phosphodiester bonds may be substituted with phosphorothioate bonds. For example, if Exonuclease III is used, the phosphodiester bonds may be substituted with phosphorothioate bonds at the 3′ ends of both strands. Optionally, a phosphorothioate may be substituted for the terminal phosphate moiety (if present) at the 3′ end of each strand.

The substrate also has a fluorophore (reporter) and a quencher. The reporter and a quencher may be located near one end one strand of the of the double stranded nucleic acid substrate. One of the fluorophore and the quencher may be attached to the end of one strand. For example, if Exonuclease III is used, one member of the fluorophore-quencher pair may be attached to the 5′ end of the target strand, while the member be attached to a nucleotide 1, 2, 3, 4, or about 5 nucleotides away from the end. For example, one strand may have a thymine-linked fluorescein at the 3^{rd} nucleotide position from the 5′ end, and an Iowa Black® quencher at the 5′ end.

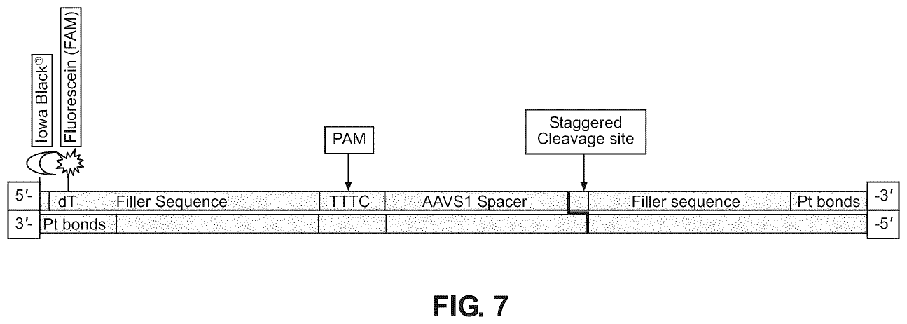

In some embodiments, the endonuclease to be tested recognizes a sequence on both strands (e.g., a palindromic sequence recognized by Type II restriction endonucleases). In some embodiments, the endonuclease to be tested recognizes a sequence on one strand (e.g., the target sequence for the CRISPR Cas endonuclease). In such embodiments, the target nucleic acid has a target strand and a non-target strand. An exemplary substrate for CRISPR Cas12a is shown in FIG. 7. The target strand of the substrate includes a targetable sequence for the endonuclease to be tested. For example, a cleavable site for Cas12a (Cpf1) is described in Zetsche, B., et al., (2015) Cpf1 is a single-RNA guided endonuclease of a Class II CRISPR-Cas system, Cell, 163:P759. As shown in FIG. 7, the substrate further includes a spacer sequence recognized by CRISPR guide nucleic acids, a fluorophore, a quencher, and exonuclease end protection such as phosphorothioate nucleotides at or near the termini of each strand.

In some embodiments, the fluorophore is placed on the target strand. In some embodiments, the fluorophore is placed on the non-target strand. In some embodiments, the fluorophore is placed on both the target strand and the non-target strand. FIG. 10 illustrates the substrate of FIG. 7 with the alternative fluorophore placement. In FIG. 10, the light and dark blocks on the top strand represent the recognition (target) sequence for CRISPR endonucleases (light block—PAM, dark block—spacer). Fluorophores are represented as stars and quenchers as dark half-moons. Dark octagons represent the exonuclease-inhibiting modifications such as phosphorothioate nucleotides.

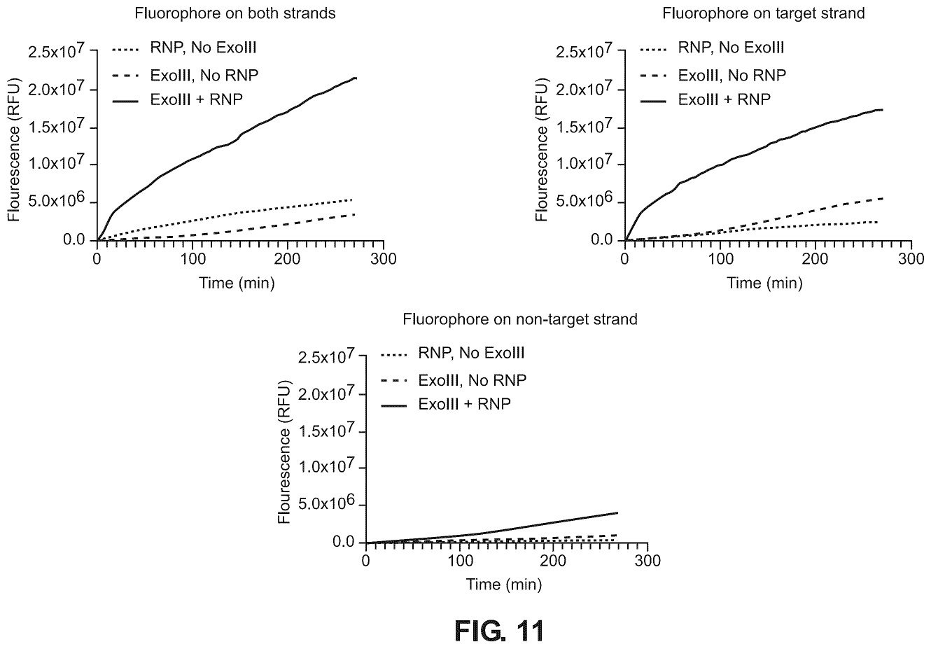

In some embodiments, fluorophore placement affects the performance of the assay (see Example 11 and FIGS. 11, 12 and 13).

The double stranded nucleic acid substrate may be prepared by combining the two strands (the target strand and the complementary non-target strand) in a reaction mixture comprising a suitable buffer (e.g., TE). For optimal annealing, the mixture may be heated to >90° C. and allowed to cool to room temperature.

In some embodiments, the effectiveness of the exonuclease protection is tested for each exonuclease intended for use in the endonuclease assay disclosed herein. For each double stranded nucleic acid substrate described herein, a control double stranded nucleic acid substrate lacking exonuclease protection is made. Both substrates are exposed to the exonuclease in a suitable buffer under suitable reaction conditions for both Exonuclease activity and fluorescence (e.g., NEBuffer 1, pH 7.0 for Exonuclease III and fluorescein) and fluorescence measured, e.g., with a fluorescence reader. If the exonuclease protection is suitable for the exonuclease, a fluorescent signal will be generated for the unprotected substrate, but not the protected substrate.

In some embodiments, the double stranded nucleic acid substrate described herein is used to detect endonuclease activity. The substrate has exonuclease protection, a fluorophore and a quencher (e.g., 1, 2, 3, 4, or about 5 phosphorothioate bonds at the 3′ ends of both strands, and thymine-linked fluorescein at the 3^{rd} nucleotide position from the 5′ end, and an Iowa Black® quencher at the 5′ end of the target strand). The substrate is contacted with the exonuclease and the endonuclease in a suitable buffer under suitable reaction conditions for exonuclease activity, endonuclease activity and fluorescence (e.g., NEBuffer 1, pH 7.0 for Exonuclease III, AsCas12a and fluorescein. Guidance for choosing a suitable buffer can be obtained from endonuclease distributors (e.g., New England Biolabs for restriction endonucleases), or from published studies, e.g., Gasiunas, G., et al., (2020) A catalogue of biochemically diverse CRISPR-Cas9 orthologs, Nature Comm. 11, Article number: 5512 doi: 10.1038 s4146 7-020-19344-1.

If the endonuclease is a nucleic acid-guided endonuclease, a nucleoprotein complex (e.g., a ribonucleoprotein complex, RNP) is assembled and the endonuclease is added to the reaction mixture in the form of the nucleoprotein complex. The nucleoprotein complex comprises the endonuclease and the nucleic acid targeting nucleic acid (NATNA), e.g., CRISPR guide RNA (crRNA) such as crRNA for Cas12a (Cpf1), suitable sequences for which can be found e.g., in Yamano T., et al., (2016) Crystal structure of Cpf1 in complex with guide RNA and target DNA, Cell 165:P949. To assemble the nucleoprotein complex, the NATNTA is incubated with the endonuclease under suitable conditions e.g., 37° C. for 10 minutes. NATNTA can be pretreated to allow for proper secondary structure formation, by heating (e.g., to 95° C. for 2 minutes) and allowed to slowly cool to room temperature.

Fluorescence of the reaction mixture is measured. For exonuclease protected substrates, a fluorescent signal is generated only when both the exonuclease and the endonuclease are present.

In some embodiments, linear range of the assay with respect to endonuclease concentration, and the concentration of the nucleic acid substrate is tested to determine the optimal range of the substrate concentration and sensitivity with respect to the endonuclease concentration.

In some embodiments, the invention is a composition for detecting activity of an endonuclease. The composition includes the nucleic acid substrate described herein further comprising a 3′-5′ exonuclease (or a 5′-3-exonuclease or both) inhibited by the structures at the 3′-ends (or the 5′-ends or both) of the substrate. The composition comprises a double-stranded nucleic acid substrate comprising: a donor fluorophore and an acceptor fluorophore (or a reporter fluorophore and a quencher fluorophore), said fluorophores forming a Fluorescence Resonance Energy Transfer (FRET) pair; a structure at each end inhibiting cleavage of the substrate by an exonuclease; and a recognition sequence for the endonuclease to be tested.

The composition may also comprise an exonuclease. Depending on the ends generated by the endonuclease, the composition comprises a 3′-5′ exonuclease or a 5′-3′ exonuclease or a mixture of both. In some embodiments, the signal is enhanced by having more than one fluorophore per substrate. In some embodiments, the exact properties of the endonuclease to be tested are not known. In such embodiments, both ends of both strands are labeled to accommodate for all possible orientations and chemistries of nucleic acid cleavage. In some such embodiments, the ends are labeled with different fluorophores emitting at different wavelengths. The emission wavelength with indicate the identity of the cleaved strand and the chemistry of cleavage. For the 3′-5′ exonuclease, the double-stranded nucleic acid substrate has a structure at each 3′-end inhibiting cleavage of the substrate by the exonuclease. For the 5′-3′ exonuclease, the double-stranded nucleic acid substrate has a structure at each 5′-end inhibiting cleavage of the substrate by the exonuclease. In some embodiments, a mixture of a 3′-5′ exonuclease and a 5′-3′ exonuclease is used. In such embodiments, both the 3′-end and the 5′-end of the substrate are protected by the exonuclease-inhibiting structure.

In some embodiments, the substrate is designed to accommodate several types of endonucleases, including endonucleases whose biochemical properties are not fully known at the time of testing. Such a substrate would have a fluorophore and a quencher pair located at or near both ends of both strands. Such doubly-labeled substrate may be utilized in a reaction mixture containing an exonuclease capable of hydrolysis in both directions (e.g., Exonuclease V or Exonuclease VII).

In some embodiments, the endonuclease to be tested by the composition is a nucleic acid-guided endonuclease. In such embodiments, a NATNA is also present in the composition. In some embodiments, the endonuclease is a CRISPR endonuclease and NATNA is a guide RNA.

In some embodiments, the endonuclease to be tested by the composition is a CRISPR Class I (CASCADE) endonuclease, the double-stranded nucleic acid substrate in the composition includes a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In the embodiments where the endonuclease to be tested by the composition is a CRISPR Class II endonuclease, the double-stranded nucleic acid substrate in the composition includes a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, and 5′-NNNACA-3′, 5′-TTN-3′, 5′-TTTN-3′ and 5′-TTTV-3′.

In some embodiments, the endonuclease to be tested by the composition is one of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In these embodiments, the double-stranded nucleic acid substrate in the composition includes a suitable recognition site.

In some embodiments, the composition comprises Exonuclease III, a double-stranded nucleic acid substrate including (i) both 3′-ends comprising one or more phosphorothioate protected dsDNA; (ii) a reporter fluorophore and (iii) a compatible fluorescence quencher positioned to quench the donor fluorescence when the double-stranded nucleic acid substrate is intact, and the endonuclease of interest.

In some embodiments, the invention provides a method suitable for use as a convenient tool for assessing, screening, testing, or comparing several endonucleases. The tool further allows to assess a set of conditions for endonuclease cleavage by allowing to determine which of the conditions permits the highest level or rate of endonuclease activity. The tool further allows for assessing endonuclease isolation and purification methods. Specifically, the tool can be applied to compare protein isolation fractions to identify the fraction containing the isolated protein. In such embodiments, modification are made to ensure that all the components, e.g., the endonuclease, the exonuclease, the NATNA (if used) are capable of being active and are at least partially protected from enzymatic degradation in the crude preparation. The tool can be rapidly applied to nascent fractions, e.g., to monitor the protein purification process. Yet further, the tool may be used to screen multiple endonuclease substrates having different sequences to quickly identify the target sequence of the endonuclease.

The methods and compositions disclosed herein can be used in a diagnostic assay. In some embodiments, the invention is a method of detecting the presence of a specific nucleic acid in a sample, wherein the nucleic acid comprises a recognition sequence for an endonuclease. In some embodiments, the sample is a patient's sample. The nucleic acid may be characteristic of a microorganism, including a virus or a bacterium. The nucleic acid may also comprise a polymorphism or a sequence whose presence related to a disease or condition to be detected in a patient.

The method involves manipulating nucleic acids from a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids that may contain nucleic acids (e.g., urine, sputum, serum, blood, or blood fractions, i.e., plasma, lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, or fecal samples). In other embodiments, the sample is a cultured sample, e.g., a tissue culture containing cells and fluids from which nucleic acids may be isolated. In some embodiments, the nucleic acids of interest present or suspected of being present in the sample come from infectious agents such as viruses, bacteria, protozoa or fungi.

In some embodiments, the method includes a preliminary amplification procedure wherein the nucleic acid in the sample is amplified via the Polymerase Chain Reaction (PCR) that generates a specific amplicon from each target nucleic acid. In some embodiments, the fluorophore, the quencher, and the end protection are incorporated into the amplicon by ligation of adapters. The formation of blunt ends, A-tailing and adaptor ligation can be performed by the methods developed e.g., in conjunction with forming a sequence library for massively parallel sequencing. In some embodiments, the fluorophore, the quencher, and the end protection are incorporated directly into the amplification primers. The excess primers or adaptors containing the fluorophore, the quencher, and the end protection may be removed via a purification procedure prior to performing an endonuclease assay. The amplicon including the fluorophore, the quencher, and the end protection is the double-stranded nucleic acid substrate used directly in the method disclosed herein.

In some embodiments, the endonuclease substrate, which is probe comprising a fluorophore, a quencher, and end protection, is hybridized to a target nucleic acid or an amplicon of the target nucleic acid in the sample. In some embodiments, the probe, the target nucleic acid or amplicon are single-stranded. In some embodiments, the probe, the target nucleic acid or amplicon are double-stranded but are rendered single-stranded prior to being hybridized to the probe. The duplex formed by the target nucleic acid hybridized to the probe including the fluorophore, the quencher, and the end protection becomes the double-stranded nucleic acid substrate used directly in the method disclosed herein.

The nucleic acid substrate formed by any of the alternative methods described above comprises a diagnostically relevant nucleic acid sequence to be interrogated. In some embodiments, the nucleic acid substrate is contacted by an endonuclease targeting the sequence of interest, e.g., a sequence characteristic of a microorganism or a sequence comprising a polymorphism or a sequence whose presence is related to a disease or condition to be detected in a patient. The endonuclease performs cleavage only if the sequence of interest comprising the endonuclease cleavage site is present in the nucleic acid substrate.

Especially advantageous in the diagnostic assay disclosed herein are the CRISPR endonucleases. A guide RNA for the CRISPR endonuclease (e.g., crRNA) may be designed to hybridize to any diagnostic sequence of interest. The sample is contacted with a probe comprising a fluorophore, a quencher, and end protection and capable of hybridizing to a target nucleic acid of diagnostic interest. The sample is further contacted with a guide RNA capable of hybridizing to the target nucleic acid. The CRSIPR endonuclease performs cleavage and fluorescence becomes detectable only if the sequence capable of hybridizing to the probe and the designed guide RNA is present in the sample.

In some embodiments, the diagnostic method is multiplex, i.e., multiple target sequences are detected in the same reaction mixture. In such embodiments, multiple nucleic acid probes are added to the sample. In some embodiments, where the endonuclease is a CRISPR endonuclease, multiple guide RNAs are also added to the sample and the same CRISPR endonuclease performs cleavage leading to generation of a detectable signal. In some embodiments, each of the different probes are labeled with a different fluorophore. In some embodiments, all or some of the different probes are labeled with the same label, e.g., one label for a set of probes hybridizing to bacterial sequences, and another label for a set of probes hybridizing to viral sequences, or one label for a set of probes hybridizing to Gram-positive bacterial sequences, and another label for a set of probes hybridizing to Gram-negative bacterial sequences.

The endonuclease and the exonuclease can be added to the sample consecutively or simultaneously. The endonuclease and the exonuclease can be added to the sample prior to the addition of the double-stranded nucleic acid substrate. The exonuclease performs strand cleavage (hydrolysis) only if the endonuclease has previously performed cleavage thus creating an exonuclease accessible terminus. Hydrolysis of the double-stranded nucleic acid substrate by the exonuclease separates the fluorophore and the quencher resulting in a detectable fluorescent signal. The presence of the fluorescent signal indicated the presence of the sequence of interest in the sample. In some embodiments, the assay includes the step of reporting that the sequence of interest (e.g., a sequence characteristic of a microorganism or polymorphism or a sequence whose presence related to a disease or condition to be detected in a patient) is present in the sample.

In some embodiments, the invention is a kit for detecting activity of an endonuclease. The kit includes an aliquot of the nucleic acid substrate described herein. The composition comprises a double-stranded nucleic acid substrate comprising: a donor fluorophore and an acceptor fluorophore (or a reporter fluorophore and a quencher fluorophore), said fluorophores forming a Fluorescence Resonance Energy Transfer (FRET) pair; a structure at each end inhibiting cleavage of the substrate by an exonuclease; and a recognition sequence for the endonuclease to be tested.

The kit may further comprise an aliquot of an exonuclease. Depending on the ends generated by the endonuclease, the kit comprises a 3′-5′ exonuclease or a 5′-3′ exonuclease. For the 3′-5′ exonuclease, the double-stranded nucleic acid substrate has a structure at each 3′-end inhibiting cleavage of the substrate by the exonuclease. For the 5′-3′ exonuclease, the double-stranded nucleic acid substrate has a structure at each 5′-end inhibiting cleavage of the substrate by the exonuclease. The kit may have both a 3′-5′ exonuclease and a 5′-3′ exonuclease. The kit may comprise a double-stranded nucleic acid substrate that has structures at both 3′-ends and 5′-ends inhibiting cleavage of the substrate by the exonuclease.

In some embodiments, the endonuclease to be tested by the kit is a nucleic acid-guided endonuclease. In such embodiments, the kit may also include an aliquot of a NATNA. In some embodiments, the endonuclease to be tested is a CRISPR endonuclease and NATNA present in the kit is a guide RNA.

In some embodiments, the endonuclease to be tested by the kit is a CRISPR Class I (CASCADE) endonuclease, the double-stranded nucleic acid substrate included in the kit includes a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′. In the embodiments where the endonuclease to be tested by the kit is a CRISPR Class II endonuclease, the double-stranded nucleic acid substrate included in the kit includes a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, and 5′-NNNACA-3′, 5′-TTN-3′, 5′-TTTN-3′ and 5′-TTTV-3′.

In some embodiments, the endonuclease to be tested by the kit is one of zinc finger nuclease (ZFN), a ZFN conjugated to Fok I, a transcription activator-like effector nuclease (TALEN), Endo TT single strand endonuclease, an Argonaute endonuclease, an Arcus endonuclease, an endoribonuclease selected from RNase III, RNase A, RNase T1, RNase A, RNase H, RNase Z and RNase P, Cas3 and Cas7-11, and a restriction endonuclease. In these embodiments, the nucleic acid substrate included in the kit includes a suitable recognition site.

In some embodiments, the kit further comprises instructions on performing the method of testing for activity of an endonuclease by a method described herein.

In some embodiments, the invention is a kit for performing a diagnostic procedure. The kit includes an aliquot of a probe capable of hybridizing to a target nucleic acid of diagnostic interest and comprising a donor fluorophore and an acceptor fluorophore (or a reporter fluorophore and a quencher fluorophore), said fluorophores forming a Fluorescence Resonance Energy Transfer (FRET) pair; a structure at each end inhibiting cleavage of the substrate by an exonuclease; and a recognition sequence for the endonuclease to be tested. The kit may further comprise an aliquot of an exonuclease and an endonuclease. The exonuclease is capable of being inhibited by the inhibitory structures present on the probe. The endonuclease is capable of binding and cleaving the duplex formed by the probe and the target sequence. In some embodiments, the endonuclease is a nucleic acid-guided endonuclease. In such embodiments, the kit may also include an aliquot of a NATNA capable of hybridizing to the target nucleic acid. In some embodiments, the endonuclease to be tested is a CRISPR endonuclease and NATNA present in the kit is a guide RNA.

In some embodiments, the kit further comprises instructions on performing the diagnostic assay described herein.

The method disclosed herein may be performed with a specialized apparatus. In some embodiments, the invention is an apparatus for detecting activity of an endonuclease comprising one or more reaction chambers for performing enzymatic reactions and a fluorescence detector. The apparatus may further comprise means for delivering and dispensing components of the reaction mixtures described herein. The apparatus may be adapted for high-throughput screening, e.g., in multiwell plates (microwell plates).

The apparatus may comprise a multi-well plate fluorescence reader or a tube fluorometer such as the ones available from Tecan, ThermoFisher Scientific (BioTek instruments), and Molecular Devices.

### EXAMPLES

### Example 1. Preparing the Double-Stranded Endonuclease Substrate

In this example, the 60-base pair double stranded nucleic acid construct was prepared with the 3′ ends of both strands protected with a series of phosphorothioate bonds, and one strand was labeled with a fluorophore and a quencher. A phosphorothioate bond was substituted for the last four phosphodiester bonds at the 3′-ends of both strands. Phosphorothioate was also substituted for the terminal phosphate moiety at the 3′-end of each strand. Additionally, the target strand had a thymine-linked fluorescein moiety incorporated at the 3^{rd} nucleotide position from the 5′-end, and an Iowa Black® quencher was attached to the 5′-end of the same strand. The dsDNA target included a well characterized model AsCas12a targetable sequence that includes Acidaminococcus sp. Cas12a enzyme (AsCas12a) cleaving site. Cleavage occurs 19-28 bp from the first thymine of the TTTC protospacer adjacent motif (PAM), which in turn is located at position 16 from the 5′-end of the substrate, and the final nucleotide of the spacer sequence located at position 39 from the 5′-end of the substrate. A second dsDNA target was prepared that was identical to the first, except it lacked end protection of any kind. Oligonucleotides with modifications were ordered from Integrated DNA Technologies (Coralville, Iowa).

The double-stranded substrate was assembled from single strands SEQ ID NO: 1 and SEQ ID NO: 2. The double stranded DNA target was prepared by combining the target strand and the complementary non-target strand oligo nucleotides at a final concentration of 50 uM each in 1×TE buffer (10 mM Tris-HCl (pH 8.0), 0.1 mM EDTA) to ensure the dsDNA was annealed properly the mixture was heated to 95° C. for 2 minutes and then slowly cooled to room temperature.

Top strand (SEQ ID NO: 1):
5′-c^{∧}at‡ggtgtcgacccatttcctggagccatctctctccttgctcgagacct
ctaagt*a*t*g-3′
Bottom strand (SEQ ID NO: 2):
5′-gcatacttagaggtctcgagcaaggagagagatggctccagga
aatgggtcgaca*c*c*a*t*g-3′

n\hat{ } quencher Iowa Black®
n‡ reporter fluorophore Fluorescein
* Phosphorothioate linkage
Underline—protospacer adjacent motif (PAM) for Cas12a
Double underline—spacer region hybridizing to a region of crRNA

A control double stranded substrate lacking phosphorothioate protection was assembled from single strands SEQ ID NO: 3 and SEQ ID NO: 4.

Top strand (no exonuclease protection) (SEQ ID NO: 3):
5′-c^{∧}at‡ggtgtcgacccatttcctggagccatctctctccttgctcgagac
ctctaag-3′
Bottom strand (no exonuclease protection) (SEQ ID NO: 4):
5′-gcatacttagaggtctcgagcaaggagagagatggctccaggaaatgggtcgaca
ccatg-3′

n\hat{ }—quencher
n‡—fluorophore
Underline—protospacer adjacent motif (PAM) for Cas12a
Double underline—spacer region hybridizing to a region of crRNA

### Example 2: Utility of an Exonuclease Coupled Fluorescent Detection

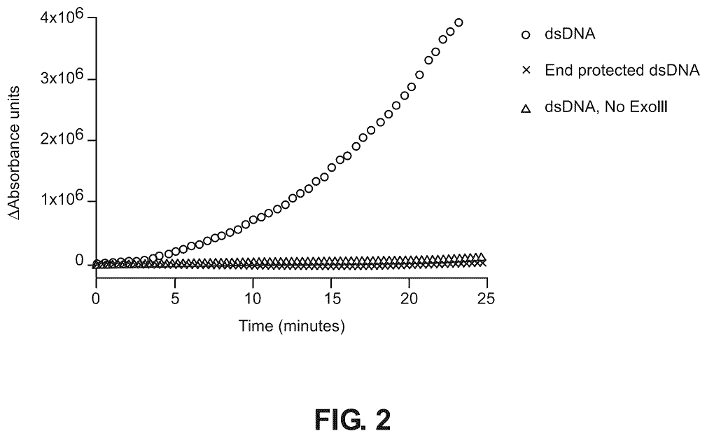

In this example, the substrates described in Example 1 were incubated at 100 nM with 0.5 U/uL Exonuclease III in 1×NEBuffer™ 1, pH 7.0 at 37° C. (both from New England Biolabs, Ipswich, Mass.). Fluorescence was monitored over time at an excitation wavelength of 485 nm and an emission wavelength of 520 nm. A fluorescent signal was generated for the unprotected substrate, but not the protected substrate. The signal was read using a Spectramax i3x plate (Molecular Devices, San Jose, Cal.). The results are shown in FIG. 2. An increase in fluorescence is observed only with unprotected substrates in the presence of Exonuclease III.

### Example 3: Cleavage of the Substrate by a Cas12a Endonuclease

In this example, we demonstrate that cleavage of a phosphorothioate protected dsDNA substrate by AsCas12a renders the phosphorothioate-protected substrate susceptible to digestion by Exonuclease III leading to a fluorescent signal that can be monitored in real time.

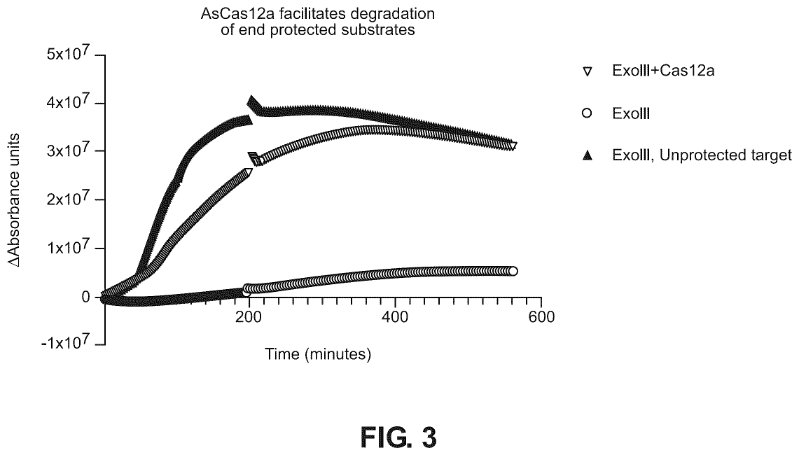

The 60 bp double stranded DNA (dsDNA) phosphorothioate-protected target described in Example 1 was used. The Cas12a ribonucleoprotein (RNP) consisting of Cas12a and a guide RNA (crRNA) was formed by incubating purified recombinant Cas12a protein at 37° C. for 10 minutes with a synthetic crRNA. Prior to RNP formation the crRNA was heated to 95° C. for 2 minutes and slowly cooled to room temperature to allow for proper secondary structure formation. The crRNA component of the RNP provided specificity to the model AsCas12a targetable sequence present in the top strand of the double-stranded substrate. The protected dsDNA target at 100 nM was incubated either with 2.5 U/uL Exonuclease III alone, or with 2.5 U/uL Exonuclease III and 112.5 nM AsCas12a RNP. The incubation was performed in 1×NEBuffer™ 1, pH 7.0. A control reaction included the unprotected dsDNA target and Exonuclease III, but no RNP. The reactions were incubated at 37° C. and fluorescence readings (Excitation: 485 nm, Emission: 520 nm) were acquired every 30 seconds for 600 minutes using a Spectramax i3x plate (Molecular Devices, San Jose, Cal.). The results are shown in FIG. 3. Rapid increase in fluorescence was observed only in the presence of Cas12a RNP and not in the no-RNP control, indicating that the end-protected dsDNA target was degraded following the Cas12a cleavage. The unprotected dsDNA target was degraded by Exonuclease III alone. During the continuous reading of fluorescence, a power interruption occurred approximately 200 minutes into the experiment. Readings were resumed quickly, but the interruption resulted a spike the fluorescent the fluorescent signal for all samples that stabilized approximately 10 minutes of readings were resumed.

### Example 4. The Linear Range of the Assay with Respect to Cas12a RNP Concentration

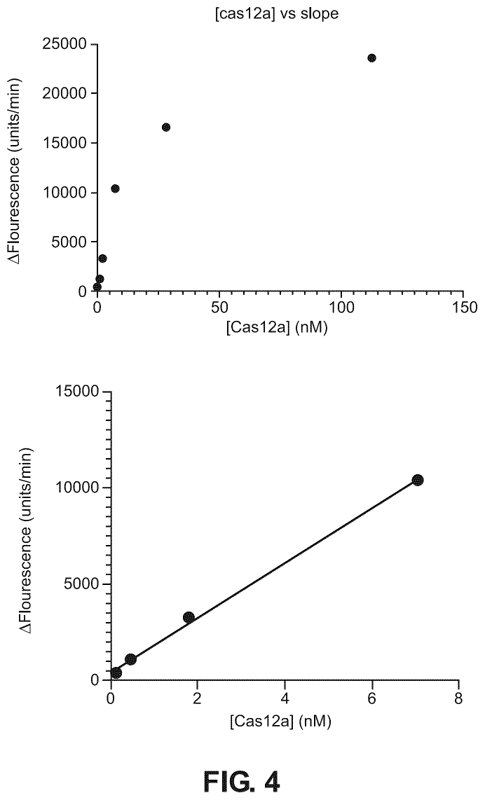

In this example, the reaction between the double stranded substrate, the Cas12a RNP and the exonuclease was performed generally as in Example 3 but using 1× Cutsmart®, pH 7.9 buffer (New England Biolabs, Ipswich, Mass.). RNP concentrations ranged from 0.11 nM to 112.5 nM. The negative controls included either no crRNA or no Cas12a RNP. The positive control included Exonuclease III only with unprotected substrate (see Example 1). Results are shown in FIG. 4. There is a direct correlation between the RNP concentration and the rate of the reaction. Reactions with lower RNP concentrations plateau at a lower fluorescence intensity. A measurable increase in fluorescence can be detected even for the 0.11 nM sample over that observed for the reaction lacking Cas12a. The results indicate a linear dose response to Cas12a concentration from 0.11 nM to 7 nM. The assay sensitivity over a wide range of Cas12a concentrations validates the utility of this quality control (QC) assay.

### Example 5. Confirmation that the Exonuclease, not Cas12a Hydrolyzes the DNA

Cas12a is a unique among endonucleases in that it possesses a non-specific exonuclease activity (“trans shredding”). In this example we demonstrate that Exonuclease III is responsible for degradation of the dsDNA target after initial cleavage by Cas12a-RNP, and not trans activity of Cas12a itself.

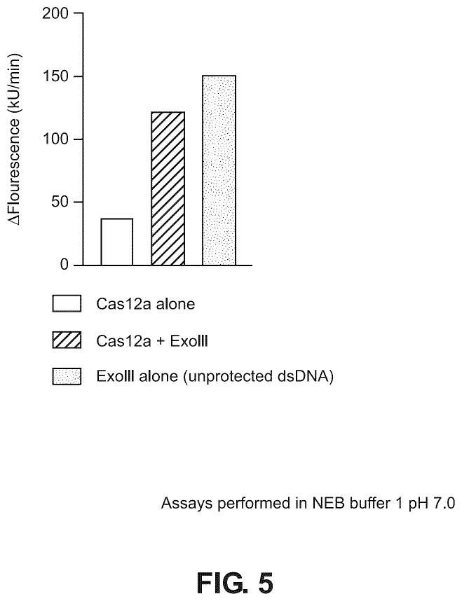

In this example, the reaction between the double stranded substrate, the Cas12a RNP and the exonuclease was performed generally as in Example 3 but using NEBuffer™ 1, pH 7.0. The concentration of the dsDNA was varied. Additionally, a control reaction contained no exonuclease. This control was included in order to determine if Cas12a was degrading the substrate on its own (without the exonuclease) through a secondary non-specific nuclease activity known as trans activity. The results are shown in FIG. 5. We observed some increase in fluorescence in the absence of Exonuclease III suggesting possible limited contribution to the overall signal from Cas12a trans activity. However, the maximum rate of fluorescence was ˜3.3-fold greater in the presence of Exonuclease III, suggesting Exonuclease III is present in sufficient concentration to ensure the reaction rate is not limited by Cas12a trans activity. The maximum rate observed was for the unprotected dsDNA substrate with Exonuclease III alone, indicating that Exonuclease III activity is not rate limiting.

### Example 6. The Linear Range of the Assay with Respect to DNA Substrate Concentration

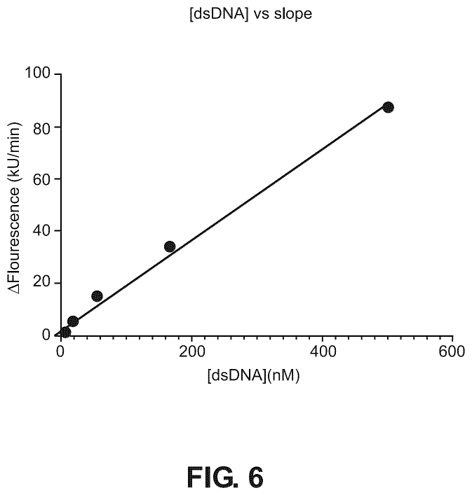

In this example, the reaction between the double stranded substrate, the Cas12a RNP and the exonuclease was performed as in Example 5. With all other reagents being kept constant, the concentration of the DNA substrate varied between 2.06 nM and 500 nM. The results are shown in FIG. 6. We observed that the assay is sensitive enough to detect down to at least 4.1 nM target dsDNA sequence and gives a linear dose response over a wide range of target dsDNA concentrations.

### Example 7 (Prophetic): Cleavage of the Substrate by a Cas9 Endonuclease

In this example, the experiment described in Example 3 is performed with a Cas9 endonuclease instead of the Cas12a endonuclease. A double stranded DNA (dsDNA) phosphorothioate-protected target is similar to SEQ ID NO: 1/SEQ ID NO: 2, except it comprises the PAM sequence recognized by Cas9. Additionally, dsDNA target harbors a fluorophore and quencher pair at the 5′ end of the non-target strand as well as the target strand. The incubation is performed in 1× Cutsmart® buffer. The Cas9 ribonucleoprotein (RNP) consisting of SpyCas9 (Streptococcus pyogenes Cas9) and a guide RNA (crRNA) is formed by incubating purified recombinant Cas9 protein at 37° C. for 10 minutes with a synthetic crRNA. The crRNA component of the RNP provides specificity to the SpyCas9 and the double-stranded substrate. The protected dsDNA target is incubated either with Exonuclease III and Cas9 RNP. One or more control reactions are included, e.g., omitting the RNP, omitting the exonuclease, or omitting the exonuclease protection. The reactions are incubated at 37° C., and fluorescence readings corresponding to the emission wavelength of the reporter fluorophore are acquired every 30 seconds for 600 minutes using a Spectramax i3x plate (Molecular Devices, San Jose, Cal.).

### Example 8 (Prophetic): Cleavage of the Substrate by a Restriction Endonuclease

In this example, the experiment described in Example 3 is performed with a restriction endonuclease, e.g., a Type II restriction endonuclease instead of the Cas12a endonuclease. A double stranded DNA (dsDNA) phosphorothioate-protected target is similar to SEQ ID NO: 1/SEQ ID NO: 2, except it comprises a recognition sequence for the restriction endonuclease to be tested. The protected dsDNA target is incubated with Exonuclease III and the restriction endonuclease in the appropriate buffer permissible for both the restriction endonuclease activity and Exonuclease III activity. One or more control reactions are included, e.g., omitting the restriction endonuclease, omitting the exonuclease, or omitting the exonuclease protection. The reactions are incubated at 37° C., and fluorescence readings corresponding to the emission wavelength of the reporter fluorophore are acquired every 30 seconds for 600 minutes using a Spectramax i3x plate (Molecular Devices, San Jose, Cal.).

### Example 9. Cleavage of the FAM Substrate by CRISPR Cas12a

In this example we designed the substrate (“FAM substrate”) shown in FIG. 7. The 60 bp target contained the protospacer adjacent motif (PAM) for Cas12a and the well-characterized AAVS1 spacer sequence. At the 5′ end upstream of the PAM the substrate has a deoxythymidine (dT)-conjugated fluorescein (FAM) paired with Iowa Black dark quencher. Both 3′-ends are protected with phosphorothioate bonds (Pt). The control substrate did not have Pt protections at the 3′-ends (“unprotected FAM substrate”).

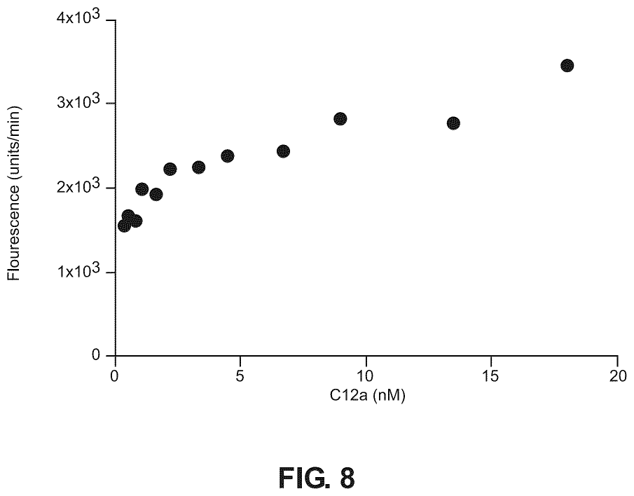

The substrate (FIG. 7) was cleaved in the reaction mixture containing the FAM substrate, Exo III, and AsCas12a ribonucleoprotein complex (RNP) at 0.42-18 nM RNP, 2.5 kU/mL Exo III and 100 nM dsDNA target in NEBuffer 1. The reaction was allowed to proceed at 37° C. and fluorescent data was gathered as shown in FIG. 8. The rates of fluorescence increase in the 50-150 minutes time period were plotted as the function of Cas12a concentration.

### Example 10. Cleavage of the TAMRA Substrate by CRISPR Cas12a

In this example we designed the substrate (“TAMRA substrate”) identical to the FAM substrate shown in FIG. 7, except TAMRA-NHS ester was used in place of the deoxythymidine (dT)-conjugated fluorescein (FAM). As with the FAM substrate (FIG. 7), both 3′-ends were protected with phosphorothioate bonds (Pt). The control substrate did not have Pt protections at the 3′-ends (“unprotected TAMRA substrate”).

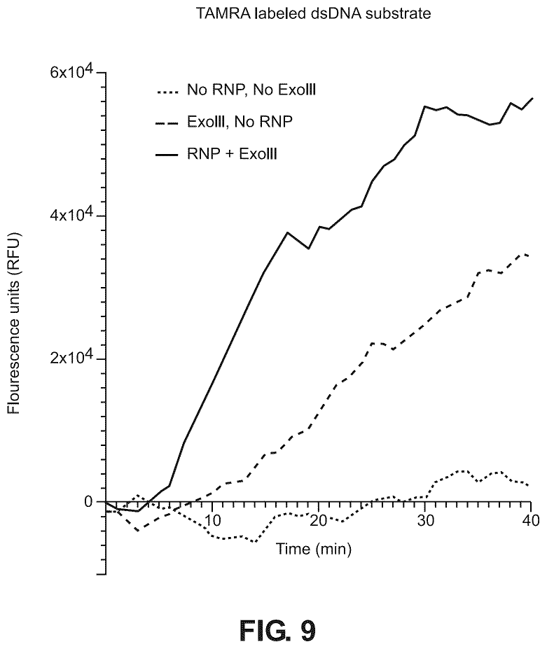

The substrate was cleaved in the reaction mixture containing the TAMRA substrate, Exo III, and AsCas12a ribonucleoprotein complex (RNP). The reaction mixture contained 18 nM RNP (or control 1×NCA buffer), 2.5 kU/mL Exo III, and 100 nM dsDNA substrate in NEBuffer 1. The reaction was allowed to proceed at 37° C. and fluorescent data was gathered as shown in FIG. 9.

### Example 11. Alternative Fluorophore Placements

In this example we designed a series of CRISPR Cas12a substrates (like the one shown in FIG. 7) but with alternative fluorophore placements. FIG. 10 shows the “non-target FAM” substrate (the same as shown in FIG. 7, having FAM on the same strand and upstream of the PAM), the “target FAM” substrate (FAM on the opposite strand from the PAM), and the “double FAM” substrate (FAM both on the same strand and the opposite strand from the PAM).

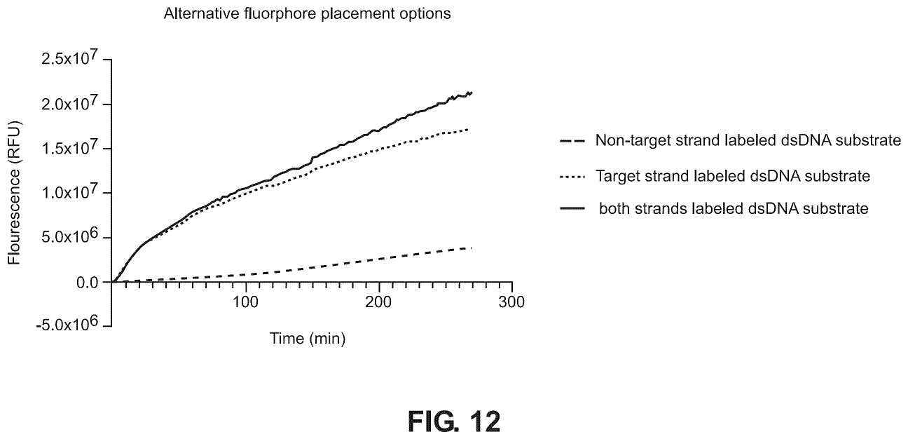

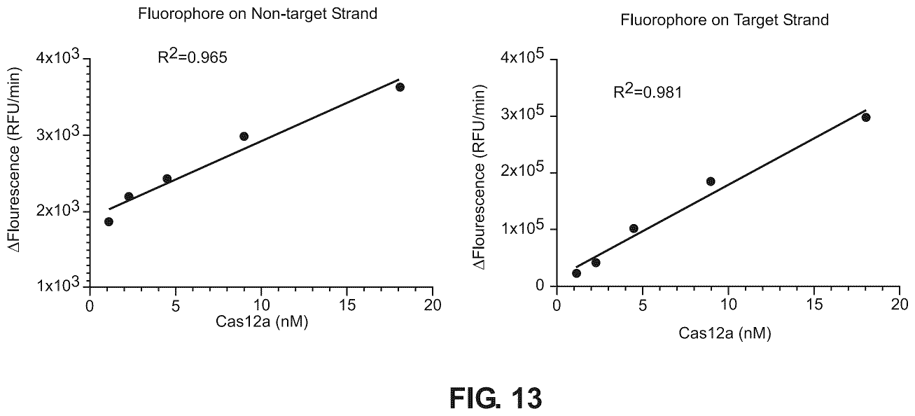

The three substrates were cleaved in reaction mixtures containing one of the three FAM substrates, Exo III and AsCas12a ribonucleoprotein complex (RNP). The reaction mixtures contained 18 nM RNP (or control 1×NCA buffer), 2.5 kU/mL Exo III, 100 nM dsDNA substrate in NEBuffer 1. The reaction was allowed to proceed at 37° C. and fluorescent data was gathered (shown in FIG. 11). The data is also presented as a comparison of all protected substrates (FIG. 12) and the rate of fluorescence change in various reaction mixtures (FIG. 13).

### Example 12. Optimizing Exonuclease Concentration

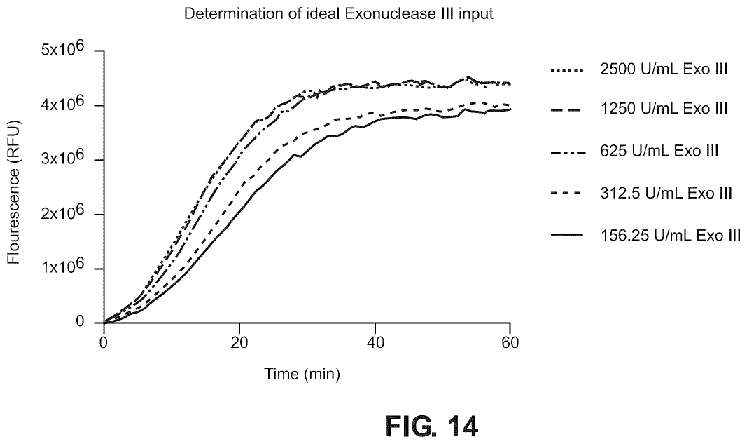

In this example we titrated the exonuclease and optimized the exonuclease concentration. The cleavage reactions were set up in NEBuffer 1 buffer and contained the “target strand” FAM substrate (FIG. 10), AsCas12a RNP at 20.25 nM RNP (2:1 cas12a:crRNA), varying amounts of ExoIII (156.25-2500 U/mL) and the FAM-labeled substrate. The control reactions contained an unprotected substrate, or no RNP, or no ExoIII. The reactions were allowed to proceed at 37° C. Results are shown in FIG. 14.

### Example 13. Cleavage with Cas9 Endonuclease

In this example we demonstrated applicability of the assay to Cas9. This example consists of validating the prophetic Example 7. As proposed in Example 7, the double stranded DNA (dsDNA) was designed according to FIG. 1 and FIG. 7. The substrate was phosphorothioate-protected and comprised the PAM sequence recognized by Cas9, the spacer sequence, the fluorophore and the quencher on the same strand as the PAM (non-target strand). In this experiment, the Cas9 guide targeted the opposite strand from the Cas12a guide.

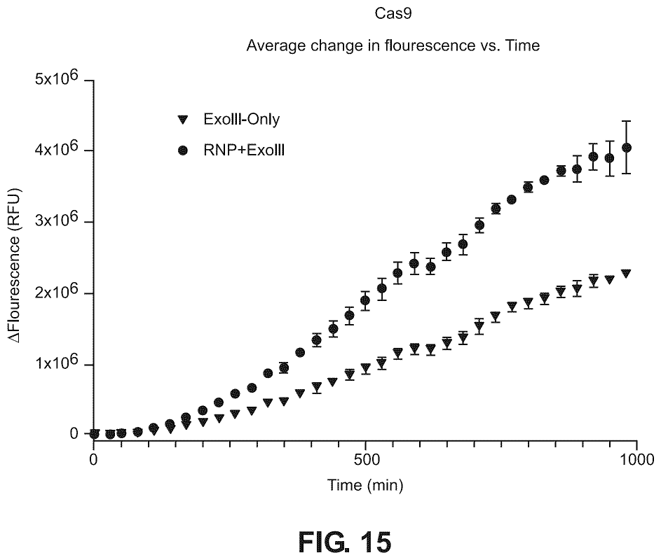

We designed a single-guide RNA (sgRNA) for the Cas9 endonuclease which combined crRNA and tracrRNA. As proposed in Example 7, the incubation is performed in 1× Cutsmart® buffer. The Cas9 ribonucleoprotein (RNP) consisting of SpyCas9 (Streptococcus pyogenes Cas9) and the guide RNA (single-guide RNA (sgRNA)) was formed by incubating purified recombinant Cas9 with the sgRNA at 37° C. for 10 minutes. The reactions included 50 nM SpyCas9, 1 kU/mL ExoIII (New England Biolabs, Ipswich, Mass.), and the dsDNA substrate at 110 nM in a 200 uL reaction volume. As proposed in Example 7, a control reaction omitting the RNP (ExoIII-only reaction) was included. The reactions are incubated at 37° C., and fluorescence readings corresponding to the emission wavelength of the reporter fluorophore are acquired every 30 minutes for 1000 minutes using a Spectramax i3x plate (Molecular Devices, San Jose, Cal.). All reactions were performed in triplicate and average results for each data point are shown in FIG. 15.

### Example 14. Cleavage with Restriction Endonucleases

In this example we demonstrated applicability of the assay to Type II restriction endonucleases. This example consists of validating the prophetic Example 8.

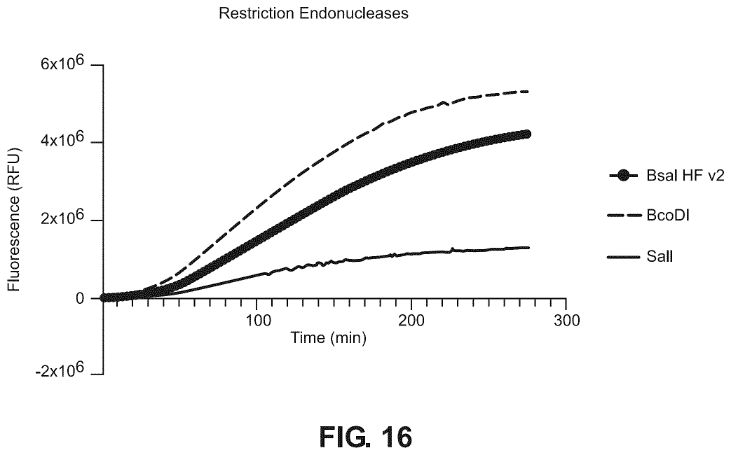

As proposed in Example 8, double stranded DNA (dsDNA) phosphorothioate-protected substrates were designed according to FIG. 1. The substrates contained recognition sequences for one of the restriction endonucleases BsaI, BcoDI and SalI. As proposed in Example 7, each substrate was incubated with Exonuclease III and the restriction endonuclease in 1× Cutsmart® buffer. Each reaction mixture comprised 300 U of one of BsaI HF v2, BcoDI and SalI (New England Biolabs) and 1.25 kU of ExoIII. The reactions are incubated at 37° C., and fluorescence readings corresponding to the emission wavelength of the reporter fluorophore are acquired every minute for 300 minutes using a Spectramax i3x plate (Molecular Devices, San Jose, Cal.). Results are shown in FIG. 16.

While the invention has been described in detail with reference to specific examples, it will be apparent to one skilled in the art that various modifications can be made within the scope of this invention. Thus, the scope of the invention should not be limited by the examples described herein, but by the claims presented below.

1-243. (canceled)

244. A composition when used for detecting activity of an endonuclease, the composition comprising a nucleic acid substrate for detecting activity of an endonuclease comprising:
(a) a donor fluorophore and an acceptor fluorophore, said fluorophores forming a Fluorescence Resonance Energy Transfer (FRET) pair;
(b) at least one structure on at least one nucleic acid strand inhibiting cleavage of the substrate by an exonuclease; and
(c) a recognition sequence and a cleavage site for an endonuclease, wherein the endonuclease is selected from the group consisting of a nucleic acid-guided endonuclease, a zinc finger nuclease (ZFN), a ZFN conjugated to Fok I, a transcription activator-like effector nuclease (TALEN), an Argonaute endonuclease, an Arcus endonuclease, an engineered endoribonuclease; Cas3 and Cas7-11 and a Type II restriction endonuclease;
wherein:
the donor fluorophore and the acceptor fluorophore are not separated by the endonuclease cleavage site; and
the composition further comprises the exonuclease.

245. The composition of claim 244, wherein the exonuclease is selected from Exonuclease I, Exonuclease III, Exonuclease V, Exonuclease VII, Exonuclease VIII, T5 Exonuclease, T7 Exonuclease, Lambda Exonuclease, Exonuclease T, BAL-31 exonuclease, RecJf exonuclease, RNase R, RNase II, RNase D, RNase T, RNase BN, RNase PH, Exoribonuclease I, and Exoribonuclease II.

246. The composition of claim 244, wherein the donor fluorophore is selected from a group consisting of 5-carboxyfluorescein (5-FAM), 6-carboxyfluorescein (6-FAM), 2′,4′,1,4,-tetrachlorofluorescein (TET), 2′,4′,5′,7′, 1,4-hexachlorofluorescein (HEX), 2′,7′-dimethoxy-4′,5′-dichloro-6-carboxyfluorescein (JOE), coumarin dyes, Alexa Fluor dyes, IRDye 800CW, Cascade Blue, Pacific Blue, Pacific Orange, Texas Red, and BODIPY® dyes.

247. The composition of claim 244, wherein the acceptor fluorophore is selected from a group consisting of tetramethyl-6-carboxyrhodamine (TAMRA), tetrapropano-6-carboxyrhodamine (ROX), DABSYL, DABCYL (4-[[4-(dimethylamino)-phenyl]-azo]-benzoic acid), Cy5 and Cy5.5, anthraquinone dyes, nitrothiazole dyes, nitroimidazole dyes, LC-Red 610, LC-Red 640, LC-Red 705, JA286, DDQ-I, DDQ-II, QSY-7, QSY-21, IRDye QC1, Iowa Black FQ, Iowa Black RQ, HEX (hexachloro-fluorescein), TET (tetrachloro-fluorescein), JOE (5′-Dichloro-dimethoxy-fluorescein), BODIPY® dyes, Eclipse Quencher (4-[[2-chloro-4-nitro-phenyl]-azo]-aniline, BHQ-1 ([(4-(2-nitro-4-methyl-phenyl)-azo)-yl-((2-methoxy-5-methyl-phenyl)-azo)]-aniline), BHQ-2 ([(4-(1-nitro-phenyl)-azo)-yl-((2,5-dimethoxy-phenyl)-azo)]-aniline), and pyridinyl-isoquinoline-dione dyes.

248. The composition of claim 244, wherein the exonuclease is selected from Exonuclease III, T5 Exonuclease, T7 Exonuclease, Lambda Exonuclease, and BAL31 Exonuclease, RNase R, RNase II, RNase D, RNase T, RNase BN, RNase PH, Exoribonuclease I, and Exoribonuclease II.

249. The composition of claim 244, wherein the nucleic acid-guided endonuclease is a CRISPR Class I (CASCADE) endonuclease and the substrate comprises a protospacer adjacent motif (PAM) consisting of a sequence selected from 5′-AAG-3′, 5′-AGG-3′, 5′-ATG-3′, 5′-GAG-3′, 5′-CAG-3′, 5′-GTG-3′, 5′-TAA-3′, 5′-TGG-3′, 5′-AAA-3′, 5′-AAC-3′, 5′-AAT-3′, 5′-ATA-3′, 5′-TAG-3′, and 5′-TTG-3′.

250. The composition of claim 244, wherein the nucleic acid-guided endonuclease is a CRISPR Cas9 endonuclease or a CRISPR Cas12a endonuclease and the substrate comprises a protospacer adjacent motif (PAM) consisting of a sequence selected from 5′-NGG-3′, 5′-NGGNG-3′, 5′-NNAAAAW-3′, 5′-NNNNGATT-3′, 5′-GNNNCNNA-3′, 5′-NNNACA-3′, 5′-TTN-3′, 5′-TTTN-3′ and 5′-TTTV-3.

251. The composition of claim 244, wherein the nucleic acid-guided endonuclease comprises an endonuclease and a nucleic acid targeting nucleic acid (NATNA) and the NATNA comprises DNA and RNA nucleotides.

252. The composition of claim 244, wherein the structure inhibiting cleavage of the substrate by an exonuclease is selected from a hairpin, a strand overhang, and a nucleic acid modification.

253. A method for detecting activity of an endonuclease in a sample, the method comprising:
(a) contacting the sample with a reaction mixture comprising the composition of claim 244 under conditions suitable for endonuclease and exonuclease cleavage of the nucleic acid substrate; and
(b) measuring fluorescence emitted by the reaction mixture as a result of endonuclease cleavage and subsequent exonuclease cleavage, wherein a change in fluorescence indicates activity of the endonuclease.

254. The method of claim 253, wherein the nucleic acid substrate or the endonuclease are in an unpurified form.

255. A kit for detecting activity of an endonuclease comprising the composition of claim 244.

256. An apparatus for detecting activity of an endonuclease by the method of claim 253 comprising a reaction chamber for performing enzymatic reactions and a fluorescence detector.

257. A method for detecting the presence of a target nucleic acid in a sample, the method comprising:
(a) contacting a sample comprising a target nucleic acid that is or has been rendered single-stranded with the composition of claim 244, wherein the nucleic acid substrate is a probe capable of hybridizing to a target nucleic acid and the probe is or has been rendered single-stranded under conditions suitable for forming a duplex between the probe and the target nucleic acid and for endonuclease and exonuclease cleavage of the substrate; and
(b) measuring fluorescence emitted by the reaction mixture, wherein a change in fluorescence indicates the presence of the target nucleic acid in the sample.

258. The method of claim 257, wherein the sample comprises a crude preparation of nucleic acids.

259. A kit for performing a diagnostic procedure consisting of detecting a target nucleic acid according to the method of claim 257, the kit comprising the composition of claim 244, wherein the target nucleic acid is selected from a sequence characteristic of a bacterium, a sequence characteristic of a virus, a sequence characteristic of a parasite, and a patient's sequence characteristic of a patient's disease or condition.

260. A method for optimizing endonuclease digestion reactions, the method comprising:
(a) preparing a series of reaction mixtures with an exonuclease and a nucleic acid substrate according to the composition of claim 244;
(b) contacting each of the series of reaction mixtures with different amounts of the endonuclease;
(c) measuring fluorescence emitted by the reaction mixture, wherein a change in fluorescence indicates activity of the endonuclease;
(d) selecting the amount of endonuclease yielding the highest fluorescence of the reaction mixture or the highest rate of increase of fluorescence of the reaction mixture as the optimal endonuclease concentration.

261. A method for optimizing CRISPR endonuclease digestion reactions, the method comprising:
(a) preparing a series of reaction mixtures with a CRISPR endonuclease, an exonuclease and a nucleic acid substrate comprising:
i. a donor fluorophore and an acceptor fluorophore, said fluorophores forming a Fluorescence Resonance Energy Transfer (FRET) pair;
ii. at least one structure on at least one strand inhibiting cleavage of the substrate by the exonuclease; and
iii. a recognition sequence for an endonuclease;
(b) contacting each of the reaction mixtures in the series with a series of nucleic acid targeting nucleic acids (NATNAs);
(c) measuring fluorescence emitted by the reaction mixture, wherein a change in fluorescence indicates activity of the endonuclease;
(d) selecting the NATNA yielding the highest fluorescence of the reaction mixture as the optimal NATNA.
